(19) United States
(12) Patent Application Publication
(10) Pub. No.: US 2009/0030033 A1
(43) Pub. Date: Jan. 29, 2009

(54) Novel Compounds 894

(75) Inventors: Jeffrey Scott Albert, Wilmington, DE (US); Cristobal Alhambra, Wilmington, DE (US); Todd Andrew Brugel, Wilmington, DE (US); Jeffrey Gilbert Varnes, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/177,259

(22) Filed: Jul. 22, 2008

Related U.S. Application Data
(60) Provisional application No. 60/951,365, filed on Jul. 23, 2007.

(51) Int. Cl. A61K 31/439; C07D 221/22; A61P 25/18; A61P 25/24; A61P 25/22

(52) U.S. Cl. 514/299; 546/112

(57) ABSTRACT

The invention relates to 2-aza-bicyclo[2.2.2]octane compounds and uses thereof. Particularly the invention relates to such compounds and their uses as pharmaceuticals in treating pyshoses such as schizophrenia and other diseases, disorders, or conditions.

This application claims benefit of priority to U.S. provisional patent application No. 60/951,365 filed Jul. 23, 2007, which is hereby incorporated by reference in its entirety.

### FIELD OF INVENTION

The present invention relates to 2-aza-bicyclo[2.2.2]octane compounds and uses thereof. Particularly the invention relates to such compounds and their uses as pharmaceuticals. More particularly the invention relates to such compounds and their uses in treating psychoses including, but are not limited to, schizophrenia, bi-polar disorder, mania and manic depression, anxiety and other cognitive diseases, disorders, or conditions. In some embodiments, the invention relates to such compounds and their uses in treating pain.

### BACKGROUND

Since the discovery of the unique behavioral effects of PCP, a number of studies have been performed to evaluate the degree of similarity between the symptoms and neurocognitive deficits induced by NMDA antagonists and those observed endogenously in schizophrenia. Studies were conducted first using PCP itself, until the drug was withdrawn from the market in the late 1960s. In those studies, PCP was found to induce not only symptoms, but also neuropsychological deficits that closely resemble those of schizophrenia. More recent studies with ketamine strongly support and extend the initial observations. Such studies led to the hypothesis that the psychotic and cognitive effects experienced by both disease sufferers and people treated with NMDA antagonists resulted from reduced NMDA receptor mediated neurotransmission. This has been termed the NMDA hypofunction hypothesis for schizophrenia. According to the hypothesis, novel treatments for schizophrenia and other psychotic diseases may result from increased NMDA activation in the central nervous system. In principle, this could be achieved by treatment with direct NMDA agonists; however, such compounds are known to cause neurotoxicity. Glycine is a requisite co-agonist for NMDA receptor, increases in its concentration may result in increased NMDA activation. The concentration of glycine is regulated by the action of the glycine transporter. Treatment with compounds that modulate the glycine transporter may increase the synaptic glycine level and thus result in NMDAr potentiation and improvement in disease symptomology.

Many people around the world continue to suffer from various psychoses and other cognitive disorders despite existing treatments. Accordingly, there is a need for new compounds, compositions, such as those that modulate the glycine transporter and methods of treatment of such diseases, disorders, or conditions employing such compounds or compositions.

### SUMMARY OF INVENTION

Some embodiments of the invention provide a compound of Formula I:

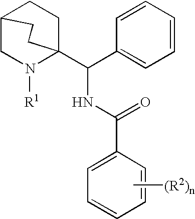

wherein:

R¹ is selected from H and C₁-C₆ alkyl;

Each R² is independently selected from halogen, —CN, C₂-C₆ alkenyl, C₂-C₆ alkynyl, C₃-C₆ cycloalkyl, —SO₂NR³R⁴, —NH₂, —S—C₁-C₆ alkyl, C₁-C₆ alkoxy, and C₁-C₆ alkyl, said C₁-C₆ alkyl and C₁-C₆ alkoxy being optionally substituted with one or more halogens;

R³ and R⁴ are each independently H or C₁-C₆ alkyl; and

n is 1, 2, or 3;

or a pharmaceutically acceptable salt thereof.

In some embodiments, each R² is independently selected from halogen, —SO₂NR³R⁴, —NH₂, and C₁-C₆ alkyl optionally substituted with one or more halogens.

In some embodiments, the invention provides a compound of Formula Ia:

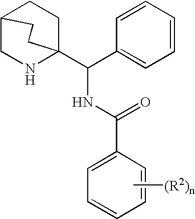

wherein:

Each R² is independently selected from halogen, —CN, C₂-C₆ alkenyl, C₂-C₆ alkynyl, C₃-C₆ cycloalkyl, —SO₂NR³R⁴, —NH₂, —S—C₁-C₆ alkyl, C₁-C₆ alkoxy, and C₁-C₆ alkyl, said C₁-C₆ alkyl and C₁-C₆ alkoxy being optionally substituted with one or more halogens;

R³ and R⁴ are each independently H or C₁-C₆ alkyl; and

n is 1, 2, or 3;

or a pharmaceutically acceptable salt thereof.

In some embodiments, the invention provides compounds of Formula Ib:

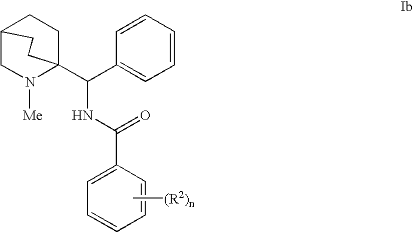

wherein:

Each R² is independently selected from halogen, —CN, C₂-C₆ alkenyl, C₂-C₆ alkynyl, C₃-C₆ cycloalkyl, —SO₂NR³R⁴, —NH₂, —S—C₁-C₆ alkyl, C₁-C₆ alkoxy, and C₁-C₆ alkyl, said C₁-C₆ alkyl and C₁-C₆ alkoxy being optionally substituted with one or more halogens;

R³ and R⁴ are each independently H or C₁-C₆ alkyl; and

n is 1, 2, or 3;

or a pharmaceutically acceptable salt thereof.

Some embodiments of the invention provide a pharmaceutical composition comprising a therapeutically effective amount of a compound according to Formula I and a pharmaceutically acceptable carrier or diluent.

Some embodiments of the invention provide a pharmaceutical composition comprising a therapeutically effective amount of a compound according to Formula Ia and a pharmaceutically acceptable carrier or diluent.

Some embodiments of the invention provide a pharmaceutical composition comprising a therapeutically effective amount of a compound according to Formula Ib and a pharmaceutically acceptable carrier or diluent.

Some embodiments provide a method of treating psychoses by administering a compound according to Formula I, Ia or Ib to a patient in need of such treatment.

Some embodiments provide a method of making a compound of Formula I, Ia, or Ib.

### DETAILED DESCRIPTION

In some embodiments, compounds disclosed herein are modulators of the Glycine Transporter I receptor, as described below.

The compounds of the invention include compounds of Formula I:

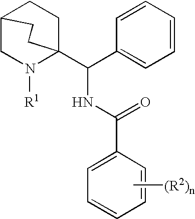

wherein:
R¹ is selected from H and C₁-C₆ alkyl;
Each R² is independently selected from halogen, —CN, C₂-C₆ alkenyl, C₂-C₆ alkynyl, C₃-C₆ cycloalkyl, —SO₂NR³R⁴, —NH₂, —S—C₁-C₆ alkyl, C₁-C₆ alkoxy, and C₁-C₆ alkyl, said C₁-C₆ alkyl and C₁-C₆ alkoxy being optionally substituted with one or more halogens; R³ and R⁴ are each independently H or C₁-C₆ alkyl; and
n is 1, 2, or 3;

or a pharmaceutically acceptable salt thereof.

In some embodiments, each R² is independently selected from halogen, —SO₂NR³R⁴, —NH₂, and C₁-C₆ alkyl optionally substituted with one or more halogens.

In some embodiments, R² is —C₁-C₆ alkoxy, more specifically, R² can be —OMe.

In some embodiments, R² is —SCH₃.

In some embodiments, R² is —CF₃.

In some embodiments, n is 1.

In some such embodiments, R² is halogen, particularly Cl, Br or F, more particularly Cl or F.

In some such embodiments, R² is C₁-C₆ alkyl, optionally substituted with one or more halogens, particularly, methyl, ethyl, or trifluoromethyl.

In some embodiments, n is 2.

In some such embodiments, R² is independently selected from the halogens, particularly Cl, Br, or F. In some embodiments, each R² is the same. In some embodiments, each R² is different.

In some embodiments, one R² is independently selected from the halogens, and the other R² is independently selected from C₁-C₆ alkyl optionally substituted with one or more halogens. In particular, the halogens are selected from Cl, Br, and F, and the C₁-C₆ alkyl is selected from methyl, and trifluoromethyl.

In some embodiments, each R² is independently selected from C₁-C₆ alkyl optionally substituted with one or more halogens. In some embodiments, each R² is the same. In some embodiments, each R² is different. In some embodiments, each R² is CF₃. In some embodiments, one R² is C₁-C₆ alkyl and the other is CF₃. In some embodiments, each R² is methyl.

In some embodiments, one or more R² is C₁-C₆ alkoxy. In some embodiments, each R² is C₁-C₆ alkoxy. In some embodiments, R² is methoxy.

In some embodiments, n is 3.

In some such embodiments, each R² is independently selected from the halogens, particularly Cl, Br, or F. In some embodiments, each R² is the same. In some embodiments, each R² is different.

In some embodiments, each R² is independently selected from the C₁-C₆ alkyl optionally substituted with one or more halogens. In some embodiments, each R² is the same. In some embodiments, each R² is different. In some embodiments, each R² is CF₃. In some embodiments, one R² is C₁-C₆ alkyl and the other is CF₃. In some embodiments, each R² is methyl.

Applicants have made compounds in racemic form, and, in some instances, also in enantiomerically pure stereochemical form. Without wishing to be bound by the theory, it appears that the R stereochemistry is favored, as they are in several instances more potent. When referring to a specific stereochemistry, “substantially pure” means greater than about 90% of one enantiomer. In some embodiments, there is 95% or greater purity. In still other embodiments, there is 98% or greater purity. In some embodiments, there is 99% or greater purity.

The “pharmaceutically acceptable salt” refers generally to non-toxic salts of the compounds of the invention which are generally prepared by reacting the free base with a suitable organic or inorganic acid. Representative salts include, but are not limited to, the following: acetate, benzenesulfonate, benzoate, bicarbonate, bisulfate, bitartrate, borate, bromide, calcium, camsylate, carbonate, chloride, clavulanate, citrate, dihydrochloride, edentate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, myethylsulfate, mutate, napsylate, nitrate, N-methylglucarnine ammonium salt, oleate, oxalate, pamoate(embonate), palmitate, pantothenate, phosphate/diphosphate, polygalacturonate, salicylate, stearate, subacetate, succinate, sulfate, sulfonate, tannate, tartrate, teoclate, tosylate, triethiodide, and valerate. Those of skill in the art will readily recognize additional pharmaceutically acceptable salts that may be employed within the scope and spirit of the invention described herein.

The term “therapeutically effective amount” when used herein means the amount of a drug or pharmaceutical agent that will elicit the biological or medical response of a tissue, system, animal or human that is being sought by a researcher or clinician. The present invention includes within its scope the use of a compound of the invention, alone or in combination with other agents, for the subject indications in a patient in need of such treatment.

“Halogen” refers to Br, Cl, F, and I.

“Cₓ-C_{y} Alkyl” as used herein refers to a straight or branched chain hydrocarbon having x to y carbon atoms.

“Cₓ-C_{y} Alkenyl” as used herein refers to a straight or branched chain hydrocarbon having x to y carbon atoms, with at least one double bond.

“Cₓ-C_{y} Alkynyl” as used herein refers to a straight or branched chain hydrocarbon having x to y carbon atoms, with at least one triple bond.

“C₃-C₆ cycloalkyl” means a carbocycle having from 3 to 6 ring carbon atoms. Such as cyclopropyl, cyclobutyl, cyclopentyl, and cyclohexane.

In some embodiments, the invention provides compounds of Formula I where R¹ is H (Formula Ia). In other embodiments, the invention provides compounds of Formula I where R¹ is methyl (Formula Ib).

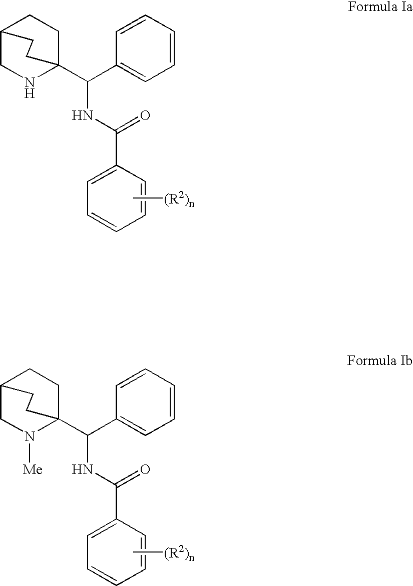

where n and R² are as described above;

or a pharmaceutically acceptable salt thereof.

In some embodiments, the invention provides compounds of either Formula Ia or Ib, wherein at least one R² is halogen.

In some embodiments, the invention provides compounds of either Formula Ia or Ib, wherein at least one R² is C₁-C₆ alkyl, optionally substituted with one or more halogen.

In some embodiments, the invention provides compounds of either Formula Ia or Ib, wherein at least one R² is methyl.

In some embodiments, the invention provides compounds of either Formula Ia or Ib, wherein at least one R² is CF₃.

In some embodiments, the invention provides compounds of either Formula Ia or Ib, wherein at least one R² is C₁-C₆ alkoxy. In some embodiments, R₂ is C₁-C₆ alkoxy substituted with one or more halogens. In some embodiments, R² is —OCF₃.

In some embodiments, the invention provides compounds of either Formula Ia or Ib, wherein at least one R² is —SCH₃.

In some embodiments, the invention provides compounds of either Formula Ia or Ib, wherein at least one R² is —SO₂NR³R⁴.

In some embodiments, the invention provides compounds of either Formula Ia or Ib, wherein each of R³ and R⁴ are hydrogen.

In some embodiments, the invention provides compounds of either Formula Ia or Ib, wherein each of R³ and R⁴ are C₁-C₆ alkyl.

In some embodiments, the invention provides compounds of either Formula Ia or Ib, wherein n is 2.

In some further embodiments, at least one R² is independently selected from —CN, C₂-C₆ alkenyl, C₂-C₆ alkynyl, C₃-C₆ cycloalkyl, —S—C₁-C₆ alkyl, and C₁-C₆ alkoxy, said C₁-C₆ alkoxy being optionally substituted with one or more halogens;

In some further embodiments, one of R² is halogen and the other R² is selected from halogen, —NH₂, —SO₂NR³R⁴, and C₁-C₆ alkyl optionally substituted with one or more halogens. In some further embodiments, the remaining R² is C₁-C₆ alkyl optionally substituted with one or more halogens. In still other embodiments, one of R² is Cl, and the other R² is C₁-C₆ alkyl optionally substituted with one or more halogens. In some such embodiments, the C₁-C₆ alkyl is methyl or CF₃.

In some further embodiments, the remaining R² is —SO₂NR³R⁴. In some further embodiments, one of R² is Cl and the other R² is —SO₂NR³R⁴ where R³ and R⁴ are H. In yet another embodiment, one of R² is Cl and R³ and R⁴ are both C₁-C₆ alkyl optionally substituted with one or more halogens. In some such embodiments, R³ and R⁴ are both methyl.

In some embodiments where n is 2, each R² is independently selected from the halogens. In some embodiments, at least one of said R² is Cl. In other embodiments, each R² is Cl.

In some embodiments, where n is 2, each R² is selected from —SO2NR³R⁴, —NH2, and C₁-C₆ alkyl optionally substituted with one or more halogens. In some embodiments, one of said R² is C₁-C₆ alkyl optionally substituted with one or more halogens. In other embodiments, each of said R² is C₁-C₆ alkyl optionally substituted with one or more halogens. In some embodiments, one of said R² is methyl or CF₃. In some embodiments, one of said R² is methyl and said other R² is CF₃. In some embodiments, each of said R² is CF₃.

In some embodiments of the invention, n is 3. In some such embodiments, at least one of R² is halogen. In further embodiments, one of R² is halogen and each remaining R² is independently selected from NH₂ and CF₃.

In some embodiments, at least two R² are independently selected from the halogens. In some such embodiments, at least two of R² are Cl. In some further embodiments, the remaining R² is —NH₂ or —CF₃.

In some embodiments, where n is 3, one of R² is Cl, one R² is F, and the remaining R² is C₁-C₆ alkyl optionally substituted with one or more halogen. In some embodiments, said remaining R² is methyl or CF₃.

In some embodiments, each R² is halogen. In some such embodiments, each R² is Cl.

Another aspect of the invention relates to pharmaceutical compositions for treating or preventing psychoses including, but not limited to, schizophrenia, bi-polar disorder, mania and manic depression, and anxiety. In some embodiments, the invention relates to compositions for treating pain. Suitable pharmaceutical compositions comprise a therapeutically effective amount of a compound of the invention, either alone or in combination with one or more other therapeutically active agents, together with an inert pharmaceutically-acceptable excipient, diluent, or carrier. Any suitable pharmaceutical dosage form may be used.

Yet another aspect of the invention relates to methods of treating a patient in need of such treatment comprising providing or administering a compound or pharmaceutical composition of the invention to the patient.

The compounds of the invention may also be used in preparing a medicament, particularly for the treatment or prophylaxis of pyschoses, such as schizophrenia, bi-polar disorder, mania and manic depression, and anxiety. In some embodiments, such medicaments can be prepared for treating pain.

Process of Making the Compounds

Compounds of Formula I may be prepared by a general method by reacting a 2-aza-bicyclo[2.2.2]octane-substituted amine 1 with a carboxylic acid 2 in the presence of a suitable coupling/dehydrating agent (or combination of reagents) such as dicyclohexylcarbodiimide and hydroxybenzotriazole, as set forth in Scheme 1. L represents a protecting group, such as carboxylic acid tert-butyl ester, when forming compounds of Formula Ia or C₁-C₆ alkyl, when forming compounds of Formula Ib. Alternatively, the substituted amine may be reacted with an acid chloride such as 2-chloro-3-trifluoromethyl-benzoyl chloride in the presence of a suitable base such as triethylamine. The resulting amide compound can then be deprotected using trifluoroacetic acid or other suitable deprotecting conditions to afford a compound of Formula Ia.

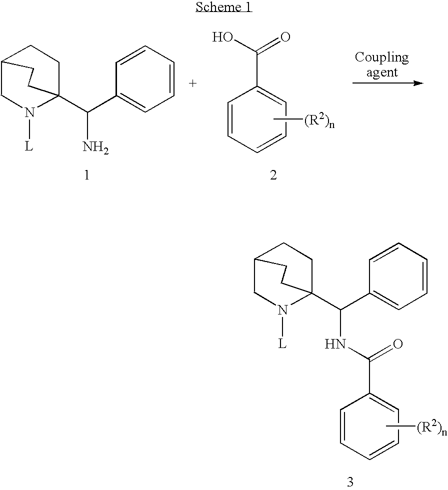

An exemplary process to form a particular compound of Formula Ia is shown in Scheme 2. An exemplary process to form the 1-(amino-phenyl-methyl)-2-aza-bicyclo[2.2.2]octane-2-carboxylic acid tert-butyl ester depicted in Scheme 2 is shown in Scheme 3.

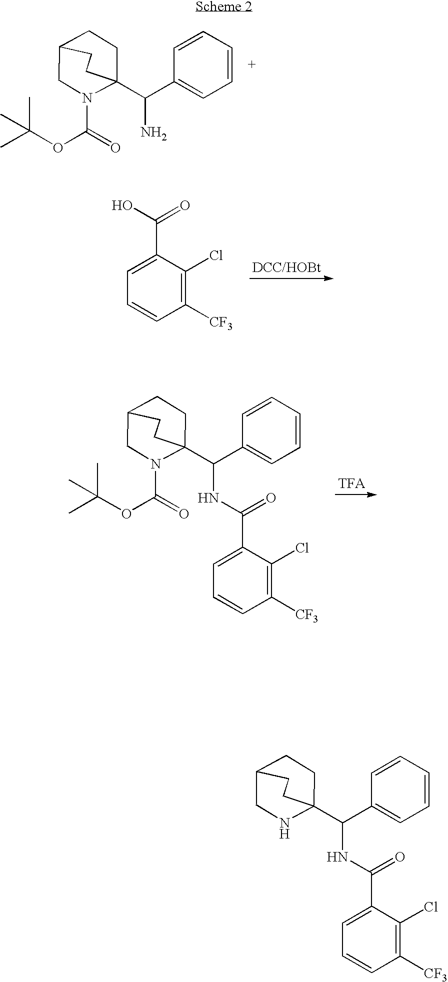

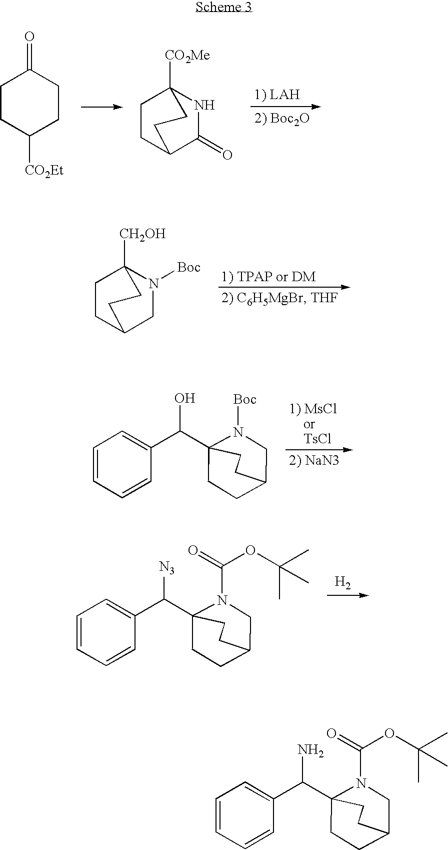

Thus, as illustrated in Scheme 2, reaction of 1-(amino-phenyl-methyl)-2-aza-bicyclo[2.2.2]octane-2-carboxylic acid tert-butyl ester with 2-chloro-3-trifluoromethyl-benzoic acid in the presence of dicyclohexylcarbodiimide and hydroxybenztriazole, or other suitable coupling agent systems will afford 1-[(2-chloro-3-methyl-benzoylamino)-phenyl-methyl]-2-aza-bicyclo[2.2.2]octane-2-carboxylic acid tert-butyl ester. This material may be reacted with trifluoroacetic acid to afford N-[(2-aza-bicyclo[2.2.2]oct-1-yl)-phenyl-methyl]-2-chloro-3-trifluoromethyl-benzamide.

Preparation of the requisite 1-(amino-phenyl-methyl)-2-aza-bicyclo[2.2.2]octane-2-carboxylic acid tert-butyl ester may be conducted as illustrated in Scheme 3. 4-Oxo-cyclohexanecarboxylic acid methyl ester (or ethyl ester) may be used to prepare 3-oxo-2-aza-bicyclo[2.2.2]octane-1-carboxylic acid ethyl ester according to the procedures described in Casabona, D.; Cativiela, C. Tetrahedron 2006, 62, 10000. This material may be reduced by lithium aluminum hydride and then protected with di-tert-butyldicarbonate to afford 1-hydroxymethyl-2-aza-bicyclo[2.2.2]octane-2-carboxylic acid tert-butyl ester. This material may be oxidized using an appropriate oxidizing agent such as tetra-N-propylammonium perruthenate (TPAP) or Dess-Martin periodinane. The resulting aldehyde (1-formyl-2-aza-bicyclo[2.2.2]octane-2-carboxylic acid tert-butyl ester) may be reacted with phenylmagnesium bromide to afford 1-(hydroxy-phenyl-methyl)-2-aza-bicyclo[2.2.2]octane-2-carboxylic acid tert-butyl ester. This material may be activated using a suitable reagent such as mesyl chloride or tosyl chloride and then reacted with sodium azide to afford 1-(azido-phenyl-methyl)-2-aza-bicyclo[2.2.2]octane-2-carboxylic acid tert-butyl ester. This material may be reduced using hydrogen in the presence of palladium to afford 1-(amino-phenyl-methyl)-2-aza-bicyclo[2.2.2]octane-2-carboxylic acid tert-butyl ester.

Some compounds of the invention may be prepared by processes analogous to those described herein and as shown in Scheme 2, by use of alternative suitable carboxylic acids (or corresponding acid chlorides) in place of 2-chloro-3-trifluoromethyl-benzoic acid to form compounds within the scope of the subject matter described herein as Formula I. Those of skill in the art will recognize that further compounds can be made by analogous methods using suitable starting materials.

Some exemplary compounds that can be made in accordance with the above scheme include:
N-(2-azabicyclo[2.2.2]octan-1-yl(phenyl)methyl)-2-chloro-3-(trifluoromethyl)benzamide;
N-(2-azabicyclo[2.2.2]octan-1-yl (phenyl)methyl)-4-chloro-3-(N,N-dimethylsulfamoyl)benzamide;
N-(2-azabicyclo[2.2.2]octan-1-yl (phenyl)methyl)-2-methyl-3-(trifluoromethyl)benzamide;
N-(2-azabicyclo[2.2.2]octan-1-yl(phenyl)methyl)-4-chloro-3-sulfamoylbenzamide;
N-(2-azabicyclo[2.2.2]octan-1-yl(phenyl)methyl)-4-amino-3,5-dichlorobenzamide;
N-(2-azabicyclo[2.2.2]octan-1-yl(phenyl)methyl)-2,6-dichloro-3-(trifluoromethyl)benzamide;
N-(2-azabicyclo[2.2.2]octan-1-yl(phenyl)methyl)-2,3-dichlorobenzamide;
N-(2-azabicyclo[2.2.2]octan-1-yl(phenyl)methyl)-3-chloro-2-methylbenzamide;
N-(2-azabicyclo[2.2.2]octan-1-yl(phenyl)methyl)-2-chloro-5-(trifluoromethyl)benzamide;
N-(2-azabicyclo[2.2.2]octan-1-yl(phenyl)methyl)-2,6-dichlorobenzamide;
N-(2-azabicyclo[2.2.2]octan-1-yl(phenyl)methyl)-2-chloro-6-fluoro-3-methylbenzamide;
N-(2-azabicyclo[2.2.2]octan-1-yl(phenyl)methyl)-2-chloro-3-methylbenzamide;
N-(2-azabicyclo[2.2.2]octan-1-yl(phenyl)methyl)-2,5-bis(trifluoromethyl)benzamide;
N-(2-azabicyclo[2.2.2]octan-1-yl(phenyl)methyl)-4-amino-3-chloro-5-(trifluoromethyl)benzamide;
N-(2-azabicyclo[2.2.2]octan-1-yl (phenyl)methyl)-3-chloro-2-fluoro-6-(trifluoromethyl)benzamide;
N-(2-azabicyclo[2.2.2]octan-1-yl(phenyl)methyl)-2,4,6-trichlorobenzamide;

and pharmaceutically acceptable salts thereof.

Compounds of Formula Ib may be prepared by a general method as follows: by reacting 3-oxo-2-aza-bicyclo[2.2.2]octane-1-carboxylic acid methyl ester with an alkylating agent to form an N-alkyl 3-oxo-2-aza-bicyclo[2.2.2]octane-1-carboxylic acid methyl ester. This material may be reduced to form a 2-alkyl-2-aza-bicyclo[2.2.2]octane-1-carboxylic acid methyl ester. The methyl ester may then be converted to the N-methyl-N-methoxy amide and reacted with an organometalic reagent to afford a (2-alkyl-2-aza-bicyclo[2.2.2]oct-1-yl)-aryl-methanone. This material may be reacted with an O-alkyl hydroxylamine to form the corresponding hydroxylamine ether. This material may be reduced to afford a C-(2-alkyl-2-aza-bicyclo[2.2.2]oct-1-yl)-C-aryl-methylamine. This material may be reacted with a carboxylic acid in the presence of a suitable coupling/dehydrating agent (or combination of reagents) such as dicyclohexylcarbodiimide and hydroxybenzotriazole. Alternatively, the substituted amine may be reacted with an acid chloride such as 2-chloro-3-trifluoromethyl-benzoyl chloride in the presence of a suitable base such as triethylamine to afford a N-[(2-alkyl-2-aza-bicyclo[2.2.2]oct-1-yl)-phenyl-methyl]-benzamide.

Thus as illustrated in Scheme 4, 3-oxo-2-aza-bicyclo[2.2.2]octane-1-carboxylic acid methyl ester may be reacted with methyl iodide to afford 2-methyl-3-oxo-2-aza-bicyclo[2.2.2]octane-1-carboxylic acid methyl ester. Reduction of this material with carbonylhydridotris(triphenylphosphine) rhodium(l) and diphenyl silane will afford 2-methyl-2-aza-bicyclo[2.2.2]octane-1-carboxylic acid methyl ester. This material may be reacted with N,O-dimethylhydroxylamine hydrochloride and trimethylaluminum to afford 2-methyl-2-aza-bicyclo[2.2.2]octane-1-carboxylic acid methoxy-methyl-amide. This material may be reacted with phenyl magnesium bromide to afford (2-methyl-2-aza-bicyclo[2.2.2]oct-1-yl)-phenyl-methanone. This material may be reacted with O-benzyl-hydroxylamine to afford (2-methyl-2-aza-bicyclo[2.2.2]oct-1-yl)-phenyl-methanone O-benzyl-oxime. This material may be reacted with lithium aluminum hydride to afford C-(2-methyl-2-aza-bicyclo[2.2.2]oct-1-yl)-C-phenyl-methylamine. This material may be reacted with 2-chloro-3-trifluoromethyl-benzoic acid in the presence of dicyclohexylcarbodiimide and hydroxybenzotriazole to afford 2-chloro-N-[(2-methyl-2-aza-bicyclo[2.2.2]oct-1-yl)-phenyl-methyl]-3-trifluoromethyl-benzamide.

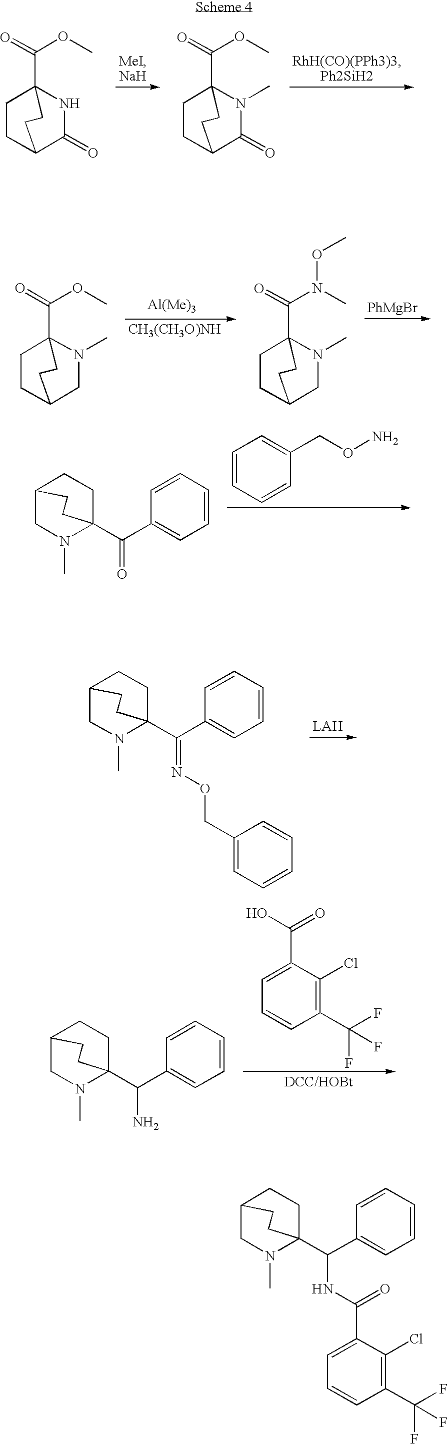

Some compounds of the invention may be prepared by processes analogous to those described herein and as shown in Scheme 3, by use of alternative suitable carboxylic acids (or corresponding acid chlorides) in place of 2-chloro-3-trifluoromethyl-benzoic acid to form compounds within the scope of the subject matter described herein as Formula Ib.

Exemplary compounds that may be made in accordance with the above schemes include:
2-chloro-N-((2-methyl-2-azabicyclo[2.2.2]octan-1-yl)(phenyl)methyl)-3-(trifluoromethyl)benzamide;
4-chloro-3-(N,N-dimethylsulfamoyl)-N-((2-methyl-2-azabicyclo[2.2.2]octan-1-yl)(phenyl)methyl)benzamide;
2-methyl-N-((2-methyl-2-azabicyclo[2.2.2]octan-1-yl)(phenyl)methyl)-3-(trifluoromethyl)benzamide;
4-chloro-N-((2-methyl-2-azabicyclo[2.2 .2]octan-1-yl)(phenyl)methyl)-3-sulfamoylbenzamide;
4-amino-3,5-dichloro-N-((2-methyl-2-azabicyclo[2.2.2]octan-1-yl)(phenyl)methyl)benzamide;
2,6-dichloro-N-((2-methyl-2-azabicyclo[2.2.2]octan-1-yl)(phenyl)methyl)-3-(trifluoromethyl)benzamide;
2,3-dichloro-N-((2-methyl-2-azabicyclo[2.2.2]octan-1-yl)(phenyl)methyl)benzamide;
3-chloro-2-methyl-N-((2-methyl-2-azabicyclo[2.2.2]octan-1-yl)(phenyl)methyl)benzamide;
2-chloro-N-((2-methyl-2-azabicyclo[2.2.2]octan-1-yl)(phenyl)methyl)-5-(trifluoromethyl)benzamide;
2,6-dichloro-N-((2-methyl-2-azabicyclo[2.2.2]octan-1-yl)(phenyl)methyl)benzamide;
2-chloro-6-fluoro-3-methyl-N-((2-methyl-2-azabicyclo[2.2.2]octan-1-yl)(phenyl)methyl)benzamide;
2-chloro-3-methyl-N-((2-methyl-2-azabicyclo[2.2.2]octan-1-yl)(phenyl)methyl)benzamide;
N-((2-methyl-2-azabicyclo[2.2.2]octan-1-yl)(phenyl)methyl)-2,5-bis(trifluoromethyl)benzamide;
4-amino-3-chloro-N-((2-methyl-2-azabicyclo[2.2.2]octan-1-yl)(phenyl)methyl)-5-(trifluoromethyl)benzamide;
3-chloro-2-fluoro-N-((2-methyl-2-azabicyclo[2.2.2]octan-1-yl)(phenyl)methyl)-6-(trifluoromethyl)benzamide;
2,4,6-trichloro-N-((2-methyl-2-azabicyclo[2.2.2]octan-1-yl)(phenyl)methyl)benzamide;

and pharmaceutically acceptable salts thereof.

This invention provides therapeutically useful compounds, processes for preparing such compounds, methods of treating diseases, disorders, or conditions using such compounds both with and without other therapeutically active agents, the use of such compounds both with and without other therapeutically active agents in the preparation of medicaments, pharmaceutical compositions containing such compounds both with and without other therapeutically active agents, and the use of such compounds both with and without other therapeutically active agents for treating various diseases, disorders or conditions.

In some embodiments, compounds described herein are useful in the treatment or prophylaxis of psychoses. Examples of psychoses include, but are not limited to, schizophrenia, bi-polar disorder, mania and manic depression, and anxiety.

In some embodiments, the invention provides a method of treating pain comprising administering a therapeutically effective amount of a compound of Formula I to a patient in need thereof.

In some embodiments, the invention provides a method of treating psychoses comprising administering a therapeutically effective amount of a compound of Formula I to a patient in need thereof.

In some embodiments, the invention provides a method of treating psychoses comprising administering a therapeutically effective amount of a compound of Formula Ia to a patient in need thereof.

In some embodiments, the invention provides a method of treating psychoses comprising administering a therapeutically effective amount of a compound of Formula Ib to a patient in need thereof.

In some embodiments, the psychosis is schizophrenia.

The invention further relates to therapies for the treatment of schizophrenia and other psychotic disorder(s) including but not limited to

Psychotic disorder(s), schizophrenia disorder(s), schizoaffective disorder(s), delusional disorder(s), brief psychotic disorder(s), shared psychotic disorder(s), and psychotic disorder(s) due to a general medical condition;

Dementia and other Cognitive Disorder(s);

Anxiety disorder(s) including but not limited to panic disorder(s) without agoraphobia, panic disorder(s) with agoraphobia, agoraphobia without history of panic disorder(s), specific phobia, social phobia, obsessive-compulsive disorder(s), stress related disorder(s), posttraumatic stress disorder(s), acute stress disorder(s), generalized anxiety disorder(s) and generalized anxiety disorder(s) due to a general medical condition;

Mood disorder(s) including but not limited to a) depressive disorder(s), including but not limited to major depressive disorder(s) and dysthymic disorder(s) and b) bipolar depression and/or bipolar mania including but not limited to bipolar I, including but not limited to those with manic, depressive or mixed episodes, and bipolar II, c) cyclothymiac's disorder(s), d) mood disorder(s) due to a general medical condition;

Sleep disorder(s);

Disorder(s) usually first diagnosed in infancy, childhood, or adolescence including but not limited to mental retardation, downs syndrome, learning disorder(s), motor skills disorder(s), communication disorders(s), pervasive developmental disorder(s), attention-deficit and disruptive behavior disorder(s), feeding and eating disorder(s) of infancy or early childhood, tic disorder(s), and elimination disorder(s);

Substance-related disorder(s) including but not limited to substance dependence, substance abuse, substance intoxication, substance withdrawal, alcohol-related disorder(s), amphetamines (or amphetamine-like)-related disorder(s), caffeine-related disorder(s), cannabis-related disorder(s), cocaine-related disorder(s), hallucinogen-related disorder(s), inhalant-related disorder(s), nicotine-related disorder(s)s, opiod-related disorder(s)s, phencyclidine (or phencyclidine-like)-related disorder(s), and sedative-, hypnotic- or anxiolytic-related disorder(s);

Attention-deficit and disruptive behavior disorder(s);

Eating disorder(s);

Personality disorder(s) including but not limited to obsessive-compulsive personality disorder(s);

Impulse-control disorder(s);

Tic disorders including but not limited to Tourette's disorder, chronic motor or vocal tic disorder; transient tic disorder;

And pain.

Many of the above conditions and disorder(s) are defined for example in the American Psychiatric Association: diagnostic and statistical manual of mental disorders, fourth edition, text revision, Washington, D.C., American Psychiatric Association, 2000.

The activity and usefulness of the compounds can be assessed in assays known to those skilled in the art. Some compounds of the invention have potency equal to or better than 1 μM (i.e. IC50≦1 μM). Some compounds in accordance with the invention have potency equal to or better than 0.5 μM (i.e. IC50≦0.5 μM). Some compounds in accordance with the invention have potency equal to or better than 0.1 μM (i.e. IC50≦0.1 μM). Still further compounds in accordance with the invention have potency equal to or better than 0.05 μM(i.e. IC50≦0.05 μM). The potency was measured in the [3H]Glycine Uptake Assay substantially as described below.

### Experimental and Analytical Details.

### [³H]Glycine Uptake Assay

### Reagents

[³H]Glycine: PerkinElmer (NET-004, [2-³H]Glycine, 53.3 Ci/mmol, 1 mCi/mL)

Cells: hGlyT1b/CHO

Cell culture medium: Ham's/F12 (Modified) (Mediatech, 10-080-CM), containing 10% FBS, 2 mM L-glutamine (Invitrogen 25030-149) and 0.5 mg/mL hygromycin (Invitrogen, 10687-010) Assay buffer 10 mM HEPES, pH 7.4, containing 150 mM NaCl, 5 mM KCl, 1.5 mM CaCl₂, 1.5 mM MgCl₂, 0.45 mg/mL L-alanine (added fresh), and 1.8 mg/mL D-glucose (added fresh).

### Procedures

Preparation of recombinant human GlyT1-CHO cells (hGlyT1-CHO). The human GlyT1b CDS (GC002087, NM_{—}006934) was cloned downstream of a CMV promoter in a bicistronic expression vector containing a hygromycin B resistance gene. CHO-K1 cells (ATCC) were transfected with the recombinant vector containing GlyT1b using Lipofectamine 2000 (Invitrogen) and cultured in Ham's/F12 media supplemented with 10% fetal bovine serum, 2 mM L-glutamine at 37° C., 5% CO₂, 90% humidity. Twenty-four hours after transfection, cells were diluted and switched to media containing 500 μg/ml hygromycin B. Antibiotic resistant cells were obtained after 21 days of culture in the presence of hygromycin B. Clonal stable cell lines were isolated by FACS single cell deposition into 96-well plates. Clonal cell lines were assessed for GlyT1b expression by measuring uptake of ³H-glycine and the clone showing the highest uptake was selected for the development of the glycine uptake assay.

Cell culture: Recombinant hGlyT1-CHO cells were cultured in cell culture medium (Ham's/F12, containing 10% FBS, 2 mM L-glutamine and 500 μg/ml hygromycin B in 175 cm² flasks until near confluence prior to use in assay.

Cell suspension: Cell medium in a cell culture flask containing near confluent cells was removed and 5 ml of cell stripper was added to submerge all cells on the surface of the culture flask. Cell stripper was removed immediately and the flask incubated in a 37° C. incubator for ˜5 min. Cells were shaken loose and suspended in 5 ml of PBS. After splitting cells to initiate a new flask(s), the cells remaining were collected by centrifugation, counted, and resuspended in assay buffer to a density of ˜2 million/mL. The cell suspension was kept at room temperature before use.

SPA and isotope mixture: WGA PTV beads were suspended in assay buffer (2 mg/ml) containing 60 nM [³H]Glycine and 20 μM unlabeled glycine and the suspension was kept at room temperature before assay.

Assay of glycine uptake: To the wells of an OptiPlate, 2 μl DMSO containing a test compound was spotted. This was followed by addition of 98 μl of cell suspension (˜1 million/ml final). After incubating cells with compound for ˜15 min, 100 μl of the SPA (200 μg/well final) and isotope mixture (30 nM isotope with 10 μM cold glycine, final) was added to initiate the glycine uptake. At 2 h, the plate was read on a TopCount to quantify SPA counts.

### HPLC

The OJH, IA and IC chiral supercritical fluid chromatography (SFC) columns were obtained from Chiral Technologies, West Chester, Pa.

### Mass Spectroscopy Method: MS1

| | |
|---|---|
| Instrumentation: | Waters Acquity SQD |
| Ionization mode: | Electrospray |
| Column: | Acquity UPLC BEH C18 2.1 × 50 mm × 1.7 um |
| Mobile phase A: | Water:Acetonitrile:Formic acid (98:2:0.1 v/v) |
| Mobile Phase B: | Water:Acetonitrile:Formic acid (2:98:0.05 v/v) |
| Gradient: | Time (% B): 0(5); 0.9(95); 1.2(95); 1.3(5); 1.4(5). |

### Mass Spectroscopy Method: MS2

| | |
|---|---|
| Instrumentation: | Agilent TOF 6210 fronted by an Agilent 1200 LC |
| Ionization mode: | Electrospray |
| Column: | Zorbax SB-C8 2.1 × 30 mm × 1.8 um |
| Mobile phase A: | Water:Acetonitrile:Formic acid (98:2:0.1 v/v) |
| Mobile Phase B: | Water:Acetonitrile:Formic acid (2:98:0.05 v/v) |
| Gradient: | Time (% B): 0(5); 1.5(95); 1.9(95); 2(5). |

When run in high-resolution mode, a reference lock mass was infused. Unless otherwise noted, mass spectroscopy method MS1 was employed.

### Mass Spectroscopy Method: MS3

| | |
|---|---|
| Instrumentation: | Waters ZMD fronted by an Agilent 1100 LC |
| Ionization mode: | Electrospray |
| Column: | Zorbax SB-C8 2.1 × 30 mm × 1.8 um |
| Mobile phase A: | Water:Acetonitrile:Formic acid (98:2:0.1 v/v) |
| Mobile Phase B: | Water:Acetonitrile:Formic acid (2:98:0.05 v/v) |
| Gradient: | Time (% B): 0(5); 3.0(90); 4.0(90); 4.5(5). |

Mass Spectroscopy method: MS4

| | |
|---|---|
| Instrumentation: | Waters/Micromass LCT TOF 6210 fronted by |
| | an Agilent 1100 LC |
| Ionization mode: | Electrospray |
| Column: | Zorbax SB-C8 2.1 × 30 mm × 1.8 um |
| Mobile phase A: | Water:Acetonitrile:Formic acid (98:2:0.1 v/v) |
| Mobile Phase B: | Water:Acetonitrile:Formic acid (2:98:0.05 v/v) |
| Gradient: | Time (% B): 0(5); 10(50); 15(90); 15.9(90), 16(5). |

### Abbreviations:

HOBT: 1-hydroxybenzotriazole

TBTU: O-benzotriazol-1-yl-N,N,N′,N′-tetramethyluronium tetrafluoroborate

### Method 1. Racemic Synthesis of Compounds of Formula Ib

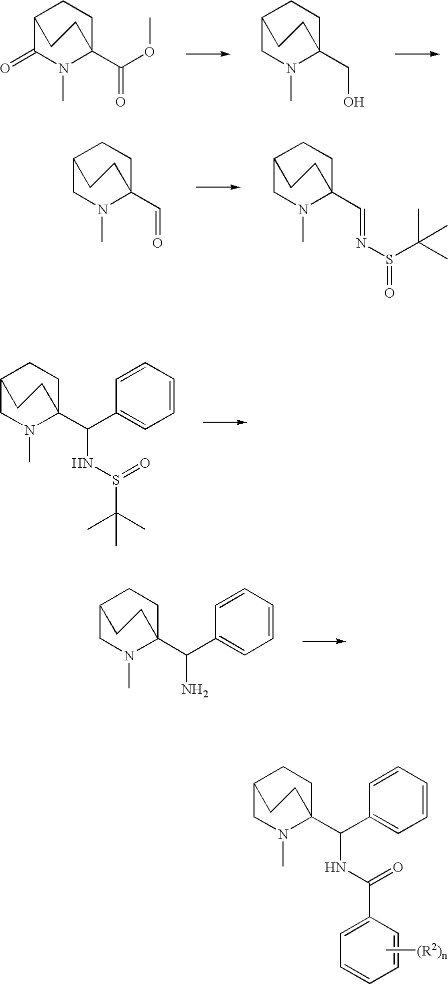

Method 1 depicts a generalized scheme suitable for racemic synthesis of compounds of Formula Ib. Those skilled in the art will readily recognize various reagents and intermediates or changes in moieties that could be used to make additional compounds of Formula Ib. R² and n can be selected as described elsewhere herein.

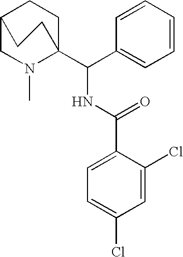

### EXAMPLE 1

### Preparation of 2,4-dichloro-N-((2-methyl-2-azabicyclo[2.2.2]octan-1-yl)(phenyl)methyl)benzamide

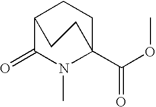

### Step A. Preparation of methyl 2-methyl-3-oxo-2-azabicyclo[2.2.2]octane-1-carboxylate from methyl 3-oxo-2-azabicyclo[2.2.2]octane-1-carboxylate

To a solution of methyl 3-oxo-2-azabicyclo[2.2.2]octane-1-carboxylate (15 g, 81.88 mmol; prepared according to the procedures of Casabona, D.; Cativiela, C. Tetrahedron, 2006, 62, 10000-10004) and iodomethane (10.24 mL, 163.75 mmol) in N,N-dimethylformamide (300 mL) at 0° C. was added 60% sodium hydride in mineral oil (3.93 g, 98.25 mmol). After stirring vigorously for 25 min, the mixture was poured into 50% aqueous sodium chloride. Ethyl acetate was added, and the layers were separated. The aqueous layer was extracted with ethyl acetate (×4), and the combined organic layers were dried over sodium sulfate, filtered, and concentrated. The resulting residue was purified by flash column chromatography (SiO₂, 0-100% ethyl acetate in hexanes) to afford an oily crystalline solid. This sample was dried under high vacuum to afford methyl 2-methyl-3-oxo-2-azabicyclo[2.2.2]octane-1-carboxylate (15.98 g, 99%) as a dry off-white crystalline solid. 1H NMR (300 MHz, chloroform-d) δ ppm 1.64-1.94 (m, 6 H), 2.06-2.22 (m, 2 H), 2.63 (quin, J=2.8 Hz, 1 H), 2.88 (s, 3 H), 3.82 (s, 3 H). ESI⁺ LCMS (M+H)⁺ 198.1.

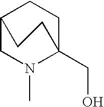

### Step B. Preparation of (2-methyl-2-azabicyclo[2.2.2]octan-1-yl)methanol from methyl 2-methyl-3-oxo-2-azabicyclo[2.2.2]octane-1-carboxylate 01305-78

To a solution of sulfuric acid (4.22 mL, 79.10 mmol) in tetrahydrofuran (2.5 mL) at 0° C. was added 2.0 M lithium aluminum hydride in tetrahydrofuran (79 mL, 158.19 mmol) dropwise. After 15 min, methyl 2-methyl-3-oxo-2-azabicyclo[2.2.2]octane-1-carboxylate (4.8 g, 24.34 mmol) was added via cannula as a solution in tetrahydruran (2.5 mL). After 3 min, the reaction was warmed to room temperature. After another 15 min, the reaction was recooled to 0° C. and quenched with sodium sulfate decahydrate. The mixture was diluted with ethyl acetate, stirred for 15 min, and filtered. The filtrate was then concentrated and filtered a final time to afford crude (2-methyl-2-azabicyclo[2.2.2]octan-1-yl)methanol (3.07 g, 81%) of 95% purity as a clear colorless oil that crystallized to form a white solid on standing. 1 H NMR (300 MHz, chloroform-d) δ ppm 1.22-1.40 (m, 2 H), 1.50-1.75 (m, 5 H), 1.79-1.94 (m, 2 H), 1.97-2.08 (m, 1 H), 2.29 (s, 3 H), 2.79 (d, J=1.1Hz, 2H), 3.34 (d, J=4.7Hz, 2H). ESI⁺ LCMS (M+H)⁺ 156.1

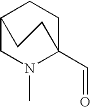

### Step C. Preparation of 2-methyl-2-azabicyclo[2.2.2]octane-1-carbaldehyde from (2-methyl-2-azabicyclo[2.2.2]octan-1-yl)methanol

To a solution of dimethylsulfoxide (5.61 mL, 79.10 mmol) in dichloromethane (99 mL) at −78° C. was added oxalyl chloride (3.46 mL, 39.55 mmol) slowly. After stirring for 30 min, (2-methyl-2-azabicyclo[2.2.2]octan-1-yl)methanol (3.07 g, 19.78 mmol) was added as a solution (20 mL) in dichloromethane via cannula. After 15 min, triethylamine (27.6 mL, 197.76 mmol) was added and the white mixture was warmed to −40° C. before being quenched with saturated aqueous sodium bicarbonate. The mixture was extracted with dichloromethane (×3) and the combined organic layers were dried over sodium sulfate, filtered and concentrated. The resulting yellow oil, 2-methyl-2-azabicyclo[2.2.2]octane-1-carbaldehyde (2.89 g, 95%) of 70% purity (containing some DMSO), was used without further purification. 1 H NMR (300 MHz, chloroform-d) δ ppm 1.48-1.79 (m, 7 H), 1.91-2.03 (m, 2 H), 2.33 (s, 3H), 2.75-2.87 (m, 2 H), 9.50 (s, 1 H). ESI⁺ LCMS (M+MeOH+H)⁺ 186.2

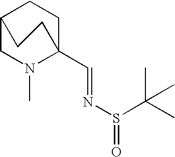

### Step D. Preparation of (E)-2-methyl-N-((2-methyl-2-azabicyclo[2.2.2]octan-1-yl)methylene)propane-2-sulfinamide from 2-methyl-2-azabicyclo[2.2.2]octane-1-carbaldehyde

To a solution of 2-methyl-2-azabicyclo[2.2.2]octane-1-carbaldehyde (1.09 g, 7.11 mmol) and tetraethoxytitanium (2.68 mL, 12.80 mmol) in tetrahydrofuran (17.78 mL) was added 2-methylpropane-2-sulfinamide (1.035 g, 8.54 mmol). After 20 h, the reaction was quenched by the dropwise addition of saturated aqueous sodium bicarbonate (1.5 mL) and subsequent dilution with ethyl acetate. The resulting white mixture was vigorously stirred for 30 min and then filtered. The filtrate was concentrated and the resulting yellow residue was purified by flash column chromatography (SiO₂, 100% ethyl acetate, then 5-30% methanol in ethyl acetate). The product fractions were concentrated and the resulting residue was taken up in ethyl acetate, filtered, and reconcentrated to afford (E)-2-methyl-N-((2-methyl-2-azabicyclo[2.2.2]octan-1-yl)methylene)propane-2-sulfinamide (1.150 g, 63.0%) as a clear colorless oil that solidified on standing. This material was stored at 0° C. prior to use. 1 H NMR (300 MHz, chloroform-d) δ ppm 1.19 (s, 9 H), 1.57-1.82 (m, 7 H), 1.92-2.16 (m, 2 H), 2.27 (s, 3H), 2.80-2.91 (m, 2 H), 7.95 (s, 1 H). ESI⁺ LCMS (M+H)⁺ 257.3.

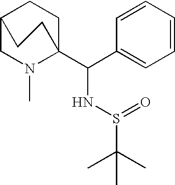

### Step E. Preparation of 2-methyl-N-((2-methyl-2-azabicyclo[2.2.2]octan-1-yl)(phenyl)methyl)propane-2-sulfinamide from (E)-2-methyl-N-((2-methyl-2-azabicyclo[2.2.2]octan-1-yl)methylene)propane-2-sulfinamide

A solution of (E)-2-methyl-N-((2-methyl-2-azabicyclo[2.2.2]octan-1-yl)methylene)propane-2-sulfinamide (0.256 g, 1.0 mmol) in THF (3.0 mL) was cooled to 0° C., and phenylmagnesium bromide (1M in THF, 2.5 mL, 2.5 mmol) was added dropwise over 5 min. The mixture was stirred for 2 hours, and then additional phenylmagnesium bromide (1 M in THF, 1.5 mL, 1.5 mmol) was added. After stirring for another 60 min, the reaction mixture was quenched with a 1:1 mixture of saturated aqueous ammonium chloride and saturated aqueous ammonium hydroxide (10 mL), extracted with ethyl acetate (×3) and the combined organic layers were washed with saturated aqueous sodium chloride, dried over magnesium sulfate, filtered, and evaporated. This afforded 2-methyl-N-((2-methyl-2-azabicyclo[2.2.2]octan-1-yl)(phenyl)methyl)propane-2-sulfinamide (310 mg, 93%) as a light yellow solid, which was used without further purification. 1 H NMR (300 MHz, chloroform-d) δ ppm 1.07-1.21 (m, 1 H), 1.25 (s, 9 H), 1.29-1.49 (m, 4 H), 1.56-1.65 (m, 2 H), 1.68-1.84 (m, 1H), 1.85-1.99 (m, 1 H), 2.44 (s, 3 H), 2.48-2.58 (m, 1 H), 3.31 (dt, J=11.0, 1.2 Hz, 1H), 4.35 (s, 1 H), 5.13 (s, 1 H), 7.19-7.35 (m, 5 H). ESI⁺ LCMS (M+H)⁺ 335.2

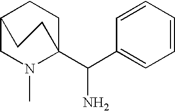

### Step F. Preparation of (2-methyl-2-azabicyclo[2.2.2]octan-1-yl)(phenyl)methanamine from 2-methyl-N-((2-methyl-2-azabicyclo[2.2.2]octan-1-yl)(phenyl)methyl)propane-2-sulfinamide

A solution of 2-methyl-N-((2-methyl-2-azabicyclo[2.2.2]octan-1-yl)(phenyl)methyl)propane-2-sulfinamide (0.390 g, 1.17 mmol) in methanol (4.0 mL) was cooled in an ice/water bath and treated with 4N hydrochloric acid in 1,4-dioxane (1.0 mL, 4.00 mmol). The mixture was stirred for 30 min, and then the cooling bath was removed. After another 30 min the reaction mixture was concentrated under reduced pressure. The residue was partitioned between water and dichloromethane, and the organic layer was discarded. The aqueous layer was made basic with concentrated ammonium hydroxide and extracted with dichloromethane (×2). The aqueous layer was then saturated with sodium chloride and further extracted with dichloromethane. The combined organic layers following basification were washed with saturated aqueous sodium chloride, dried over sodium sulfate, filtered, and concentrated. The resulting oil was vacuum dried at ambient temperature for 30 min to afford (2-methyl-2-azabicyclo[2.2.2]octan-1-yl)(phenyl)methanamine (0.263g, 98%) of 95% purity. 1 H NMR (300 MHz, chloroform-d) δ ppm 1.00-1.13 (m, 1 H), 1.29-1.47 (m, 3 H), 1.48-1.70 (m, 5 H), 1.72-1.86 (m, 1 H), 1.97-2.08 (m, 1 H), 2.43-2.49 (m, 1 H), 2.45 (s, 3 H), 3.28 (dt, J=10.6, 2.4Hz, 1H), 4.04 (s, 1 H), 7.19-7.37 (m, 5 H). ESI⁺ LCMS (M+H)⁺ 231.2.

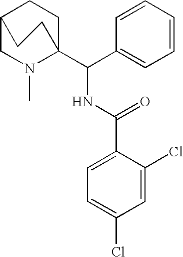

### Step G. Preparation of 2,4-dichloro-N-((2-methyl-2-azabicyclo[2.2.2]octan-1-yl)(phenyl)methyl)benzamide from (2-methyl-2-azabicyclo[2.2.2]octan-1-yl)(phenyl)methanamine

A mixture of (2-methyl-2-azabicyclo[2.2.2]octan-1-yl)(phenyl)methanamine (0.160 g, 0.69 mmol), 2,4-dichlorobenzoic acid (0.159 g, 0.83 mmol), and HOBT-hydrate (0.128g, 0.84 mmol) in N,N-dimethylformamide (6.0 mL) was treated with TBTU (0.268g, 0.83 mmol) and diisopropylamine (0.32 mL, 1.84 mmol). The mixture was stirred at ambient temperature for 16 h, and then the majority of the N,N-dimethylformamide was removed under high vacuum at 30° C. The concentrate was partitioned between aqueous potassium carbonate and dichloromethane, and the layers were separated. The organic layer was washed with water and then saturated aqueous sodium chloride, dried over sodium sulfate, filtered, and concentrated. The resulting residue was purified by flash column chromatography (SiO₂, 5% 2M ammonia in methanol in dichloromethane) to afford 2,4-dichloro-N-((2-methyl-2-azabicyclo[2.2.2]octan-1-yl)(phenyl)methyl)benzamide (0.251 g, 90%) of 95% purity as a white foam solid. Alternately, this material could be prepared by reacting (2-methyl-2-azabicyclo[2.2.2]octan-1-yl)(phenyl)methanamine in dichloromethane with 2,4-dichlorobenzoyl chloride and triethylamine to afford the same product. 1 H NMR (300 MHz, chloroform-d) δ ppm 1.30-1.79 (m, 7 H), 1.88-2.04 (m, 1H), 2.34 (s, 3H), 2.49 (d, J=10.7Hz, 1H), 2.80 (s, 1H), 3.27 (d, J=10.9 Hz, 1 H), 4.80 (d, J=3.9 Hz, 1 H), 7.19-7.35 (m, 6H), 7.44 (d, J=2.0Hz, 1H), 7.50 (br. s., 1 H), 7.61 (d, J=8.3 Hz, 1 H). ESI⁺ LCMS (M+H)⁺ 403.1329, 405.1308.

Method 2. Preparation of compounds of Formula Ib by Chiral Resolution of a Final Product

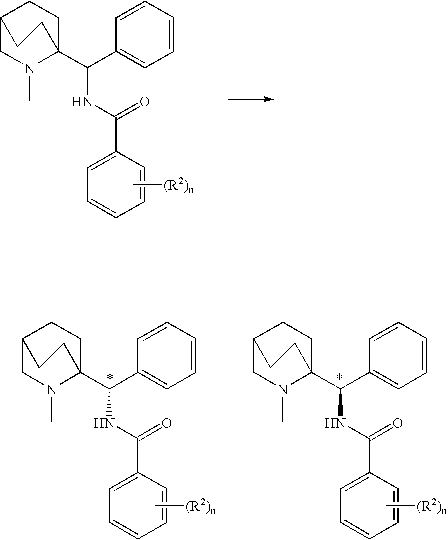

Method 2 depicts a generalized scheme suitable for preparation of compounds of Formula Ib by chiral resolution of a final product. Those of skill in the art will readily recognize various reagents and intermediates or changes in moieties that could be used to make additional compounds of Formula Ib. R² and n can be selected as described elsewhere herein.

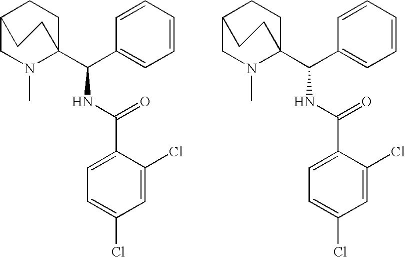

### EXAMPLES 2 AND 3

### Preparation of (R)-2,4-dichloro-N-((2-methyl-2-azabicyclo[2.2.2]octan-1-yl)(phenyl)methyl)benzamide citric acid salt and (S)-2,4-dichloro-N-((2-methyl-2-azabicyclo[2.2.2]octan-1-yl)(phenyl)methyl)benzamide citric acid salt

Racemic 2,4-dichloro-N-((2-methyl-2-azabicyclo[2.2.2]octan-1-yl)(phenyl)methyl)benzamide was resolved under supercritical fluid chromatography conditions (liquid CO₂) on a ChiralPak IC column using 30% methanol containing 0.5% dimethylethylamine to afford (R)-2,4-dichloro-N-((2-methyl-2-azabicyclo[2.2.2]octan-1-yl)(phenyl)methyl)benzamide and (S)-2,4-dichloro-N-((2-methyl-2-azabicyclo[2.2.2]octan-1-yl)(phenyl)methyl)benzamide. These compounds were dissolved in 10% methanol in dichloromethane, treated with 1.0 equiv of citric acid monohydrate in methanol and concentrated. The resulting residues were lyopholized from 1:1 acetonitrile in water to afford (R)-2,4-dichloro-N-((2-methyl-2-azabicyclo[2.2.2]octan-1-yl)(phenyl)methyl)benzamide citric acid salt and (S)-2,4-dichloro-N-((2-methyl-2-azabicyclo[2.2.2]octan-1-yl)(phenyl)methyl)benzamide citric acid salt as white solids. Relative Stereochemistry: In general, the absolute stereochemistry of individual isomers obtained in this manner was not determined; therefore arbitrary designations were used (R*, S*). However, in the case of the 2,4-dichlorobenzamides, the R* enantiomer was also prepared using Method 3 employing (R)-(2-methyl-2-azabicyclo[2.2.2]octan-1-yl)(phenyl)methanamine (Example 4, see below), and the two compounds were proven to have identical retention times under SFC conditions described above. (R)-2,4-dichloro-N-((2-methyl-2-azabicyclo[2.2.2]octan-1-yl)(phenyl)methyl)benzamide citric acid salt: 1 H NMR (300 MHz, DMSO-d₆) δ ppm 1.20-1.35 (m, 1 H), 1.36-1.73 (m, 5 H), 1.81 (br. s., 1 H), 2.02-2.17 (m, 2 H), 2.17-2.32 (m, 1 H), 2.45-2.63 (m, 4 H), 2.80 (s, 3 H), 2.88-2.99 (m, 1 H), 3.40-3.58 (m, 1 H), 5.38 (d, J=9.3 Hz, 1 H), 7.27-7.42 (m, 3 H), 7.42-7.60 (m, 4 H), 7.67 (d, J=1.5 Hz, 1 H), 9.08 (d, J=9.4 Hz, 1 H). ESI⁺ LCMS (M-citrate)⁺ 403.1340, 405.1315. (S)-2,4-dichloro-N-((2-methyl-2-azabicyclo[2.2.2]octan-1-yl)(phenyl)methyl)benzamide citric acid salt: 1 H NMR (300 MHz, DMSO-d₆) δ ppm 1.19-1.35 (m, 1 H), 1.35-1.73 (m, 5H), 1.76-1.86 (m, 1 H), 2.01-2.16 (m, 2 H), 2.17-2.31 (m, 1 H), 2.45-2.64 (m, 4 H), 2.79 (s, 3H), 2.87-2.99 (m, 1 H), 3.39-3.57 (m, 1 H), 5.37 (d, J=9.5 Hz, 1 H), 7.26-7.42 (m, 3 H), 7.42-7.60 (m, 4 H), 7.67 (d, J=1.8 Hz, 1 H), 9.07 (d, J=9.5 Hz, 1 H). ESI⁺ LCMS (M-citrate)⁺ 403.1313, 405.1307.

### Method 3. Preparation of Compounds of Formula Ib by Chiral Resolution of an Intermediate

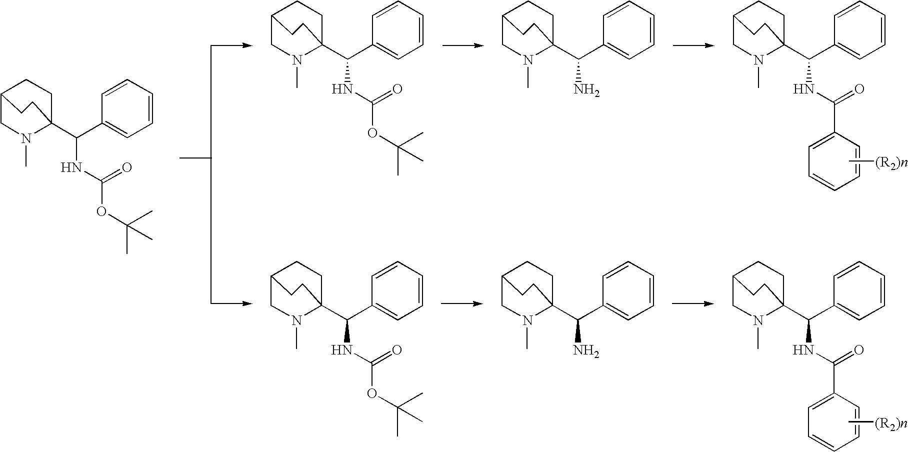

Method 3 depicts a generalized scheme suitable for preparation of of compounds of Formula Ib by chiral resolution of an intermediate. Those of skill in the art will readily recognize various reagents and intermediates or changes in moieties that could be used to make additional compounds of Formula Ib. R² and n can be selected as described elsewhere herein.

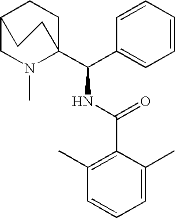

### EXAMPLE 4

### Preparation of (R)-2,6-dimethyl-N-((2-methyl-2-azabicyclo[2.2.2]octan-1-yl(phenyl)methyl)benzamide

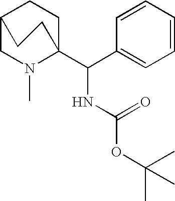

### A. Preparation of tert-butyl(2-methyl-2-azabicyclo[2.2.2]octan-1-yl)phenyl)methylcarbamate from (2-methyl-2-azabicyclo[2.2.2]octan-1-yl)(phenyl)methanamine

A mixture of (2-methyl-2-azabicyclo[2.2.2]octan-1-yl)(phenyl)methanamine (1.27 g, 5.51 mmol; Example 1, Step F), sodium bicarbonate (4.26 g, 50.71 mmol), dioxane (15.0 mL), and water (15.0 mL) was cooled in an ice/water bath. The vigorously stirring solution was treated with a solution of di-t-butyl-dicarbonate (1.25 g, 5.73 mmol) in dioxane (2 mL). After 5 minutes, the cooling bath was removed and the mixture stirred at ambient temperature for 2 hours. Additional di-t-butyl-dicarbonate (1.25 g, 5.73 mmol) was added and the mixture stirred at ambient temperature for another 16 h. Additional sodium bicarbonate (2.0 g) and di-t-butyldicarbonate (1.3 g) were added and stirring was continued for 5 h. The reaction mixture was then partitioned between water and ethyl acetate. The layers were separated, the aqueous layer was saturated with sodium chloride and further extracted with ethyl acetate. The combined organic layers were washed with saturated aqueous sodium chloride, dried over magnesium sulfate, filtered, and evaporated. The resulting residue was purified by flash column chromatography (SiO₂, 5% 2M ammonia in methanol in dichloromethane to afford tert-butyl(2-methyl-2-azabicyclo[2.2.2]octan-1-yl)(phenyl)methylcarbamate as a foam solid (1.51 g, 83%). 1 H NMR (300 MHz, chloroform-d) δ ppm 1.04-1.77 (m, 17 H), 1.79-1.93 (m, 1 H), 2.34 (s, 3 H), 2.39-2.51 (m, 1 H), 3.26 (dt, J=10.5, 2.2Hz, 1H), 4.38 (br. s., 1H), 5.75 (br. s., 1 H), 7.14-7.31 (m, 5 H). ESI⁺ LCMS (M+H)⁺ 331.2385.

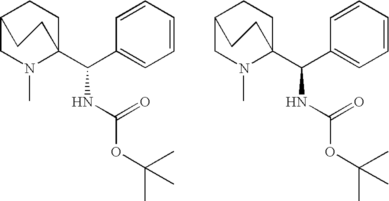

### Step B. Preparation of (S)-tert-butyl(2-methyl-2-azabicyclo[2.2.2]octan-1-yl)(phenyl)methylcarbamate and (R)-tert-butyl(2-methyl-2-azabicyclo[2.2.2]octan-1-yl)(phenyl)methylcarbamate from tert-butyl(2-methyl-2-azabicyclo[2.2.2]octan-1-yl)(phenyl)methylcarbamate

Tert-butyl(2-methyl-2-azabicyclo[2.2.2]octan-1-yl)(phenyl)methylcarbamate (4.40 g, 13.3 mmol) was resolved using an IA column and supercritical fluid chromatography conditions (liquid CO₂) employing isocratic 8.5% methanol containing 0.5% dimethylethylamine. This afforded (S)-tert-butyl (2-methyl-2-azabicyclo[2.2.2]octan-1-yl)(phenyl)methylcarbamate (1.95 g, 44%) of 95% purity and (R)-tert-butyl(2-methyl-2-azabicyclo[2.2.2]octan-1-yl)(phenyl)methylcarbamate (2.02 g, 46%) of 95% purity as a white solids. (S)-tert-butyl(2-methyl-2-azabicyclo[2.2.2]octan-1-yl)(phenyl)methylcarbamate: 1H NMR (300 MHz, chloroform-d) δ ppm 1.17-1.77 (m, 17 H), 1.79-1.93 (m, 1 H), 2.34 (s, 3 H), 2.42-2.52 (m, 1 H), 3.18-3.34 (m, 1 H), 4.39 (br. s., 1 H), 5.70-5.86 (m, 1 H), 7.10-7.40 (m, 5 H). ESI⁺ LCMS (M+H)⁺ 331.2371. (R)-tert-butyl(2-methyl-2-azabicyclo[2.2.2]octan-1-yl)(phenyl)methylcarbamate: 1H NMR (300 MHz, chloroform-d) δ ppm 1.17-1.78 (m, 17 H), 1.79-1.94 (m, 1 H), 2.34 (s, 3 H), 2.41-2.56 (m, 1 H), 3.27 (d, J=10.5 Hz, 1 H), 4.39 (br. s., 1 H), 5.77 (br. s., 1 H), 7.15-7.39 (m, 5 H). ESI⁺ LCMS (M+H)⁺ 331.2377. Chiral analytical supercritical fluid (CO₂) chromatography was carried out using a 4.6×250 mm ChiralPak IA column with a modifier composed of methanol containing 0.3% isopropyl amine. The flow rate was 2.37 mL/min with the following gradient: isocratic hold at 5% modifier for 1 min, then ramping at 5% per minute to 50% modifier, then holding at this mixture for 5 minutes. Using these conditions, the retention times for (S)-tert-butyl(2-methyl-2-azabicyclo[2.2.2]octan-1-yl)(phenyl)methylcarbamate and (R)-tert-butyl(2-methyl-2-azabicyclo[2.2.2]octan-1-yl)(phenyl)methylcarbamate were 5.5 and 5.9 minutes, respectively.

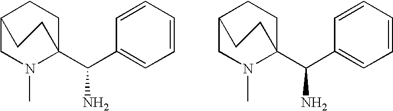

### Step C. Preparation of (S)-(2-methyl-2-azabicyclo[2.2.2]octan-1-yl)(phenyl)methanamine and (R)-(2-methyl-2-azabicyclo[2.2.2]octan-1-yl)(phenyl)methanamine from (S)-tert-butyl(2-methyl-2-azabicyclo[2.2.2]octan-1-yl)(phenyl)methylcarbamate and (R)-tert-butyl(2-methyl-2-azabicyclo[2.2.2]octan-1-yl)(phenyl)methylcarbamate.

Separate solutions of the above amines in methanol (6.11 mmol) were treated with aqueous hydrochloric acid (3N, 12.0 mL, 36.00 mmol) and concentrated aqueous hydrochloric acid (12.0 M, 3.0 mL, 36.00 mmol). The mixtures were stirred at ambient temperature for 2 hours and then concentrated under reduced pressure. The resulting residues were reconcentrated from methanol (×2; water bath temp: 45-50° C.) and then partitioned between water and dichloromethane. The layers were separated, and the organic layer was discarded. The aqueous layer was made basic with saturated aqueous ammonium hydroxide and then extracted with dichloromethane (×2). The aqueous layer was saturated with sodium chloride and further extracted with dichloromethane. The combined organic layers were washed with saturated aqueous sodium chloride, dried over magnesium sulfate, filtered, and concentrated. The resulting oils were dried under vacuum for 30 min to afford (S)-(2-methyl-2-azabicyclo[2.2.2]octan-1-yl)(phenyl)methanamine and (R)-(2-methyl-2-azabicyclo[2.2.2]octan-1-yl(phenyl)methanamine as light yellow solids (90% yields for both). (S)-(2-methyl-2-azabicyclo[2.2.2]octan-1-yl)(phenyl)methanamine: 1H NMR (300 MHz, chloroform-d) δ ppm 0.97-1.13 (m, 1 H), 1.29-1.46 (m, 3 H), 1.47-1.72 (m, 6 H), 1.72-1.86 (m, 1 H), 1.95-2.08 (m, 1 H), 2.41-2.52 (m, 1 H), 2.45 (s, 3 H), 3.28 (dt, J=10.6, 2.5 Hz, 1 H), 4.04 (s, 1 H), 7.14-7.41 (m, 5 H). ESI⁺ LCMS (M+H)⁺ 231.1859. (R)-(2-methyl-2-azabicyclo[2.2.2]octan-1-yl)(phenyl)methanamine: 1H NMR (300 MHz, chloroform-d) δ ppm 0.94-1.14 (m, 1 H), 1.28-1.47 (m, 3 H), 1.48-1.71 (m, 5 H), 1.72-1.87 (m, 1 H), 1.96-2.09 (m, 1 H), 2.43-2.52 (m, 1 H), 2.46 (s, 3 H), 3.28 (dt, J=10.6, 2.5 Hz, 1 H), 4.04 (s, 1 H), 7.13-7.37 (m, 5 H). ESI⁺ LCMS (M+H)⁺ 231.1858.

Absolute Stereochemical Configuration: The absolute chiral form of the two amines above was established through the synthesis of 1-((1S,4R)-7,7-dimethyl-2-oxobicyclo[2.2.1]heptan-1-yl)-N-((S)-(2-methyl-2-azabicyclo[2.2.2]octan-1-yl)(phenyl)methyl)methanesulfonamide, prepared by reacting presumed (S)-(2-methyl-2-azabicyclo[2.2.2]octan-1-yl)(phenyl)methanamine with excess ((1S,4R)-7,7-dimethyl-2-oxobicyclo[2.2.1]heptan-1-yl)methanesulfonyl chloride and triethyl amine in dichloromethane for 16 h. 1H NMR (300 MHz, chloroform-d) δ ppm 0.75 (s, 3 H), 1.05 (s, 3 H), 1.08-1.22 (m, 1 H), 1.24-1.75 (m, 10 H), 1.80 (d, J=18.3 Hz, 1 H), 1.84-2.04 (m, 3 H), 2.26 (dt, J=18.3, 4.0 Hz, 1 H), 2.32-2.43 (m, 1 H), 2.46 (s, 3 H), 2.55 (dt, J=11.2, 2.7 Hz, 1 H), 2.73 (d, J=14.8 Hz, 1 H), 2.97 (d, J=15.0 Hz, 1 H), 3.26 (d, J=11.2 Hz, 1 H), 4.51 (s, 1 H), 7.27-7.41 (m, 5 H). ESI⁺ LCMS (M+H)⁺ 445.4.

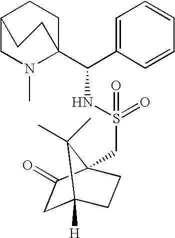

### 1-((1S,4R)-7,7-dimethyl-2-oxobicyclo[2.2.1]heptan-1-yl)-N-((S)-(2-methyl-2-azabicyclo[2.2.2]octan-1-yl)(phenyl)methyl)-methanesulfonamide

Cooling a solution of this sulfonamide in hot hexanes containing just enough acetone to solublize (˜5%) afforded crystals along the edges of the solution, which, when subjected to single crystal x-ray diffraction, proved the previously arbitrarily assigned (S) enantiomer to in fact have this very structure. The opposite enantiomer was assigned the (R) stereochemistry. Either enantiomer can be carried on as described in Step D to afford desired products.

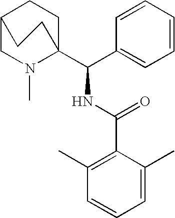

### Step D. Preparation of (R)-2,6-dimethyl-N-((2-methyl-2-azabicyclo[2.2.2]octan-1-yl)(phenyl)methyl)benzamide from (R)-(2-methyl-2-azabicyclo[2.2.2]octan-1-yl)(phenyl)methanamine

The desired compound was prepared according to the procedure of Example 1, Step G, substituting 2,6-dmethylbenzoic acid for 2,4-dichlorobenzoic acid and (R)-(2-methyl-2-azabicyclo[2.2.2]octan-1-yl)(phenyl)methanamine for (2-methyl-2-azabicyclo[2.2.2]octan-1-yl)(phenyl)methanamine. 1H NMR (300 MHz, chloroform-d) δ ppm 1.18-1.73 (m, 8 H), 1.85-2.04 (m, 1 H), 2.34 (s, 6 H), 2.41 (s, 3 H), 2.44-2.55 (m, 1 H), 3.11-3.26 (m, 1 H), 4.90 (d, J=4.4 Hz, 1 H), 6.80 (br. s., 1 H), 6.96-7.08 (m, 2 H), 7.10-7.20 (m, 1 H), 7.21-7.39 (m, 5 H).). ESI⁺ LCMS (M+H)⁺ 363.2425.

### Method 4. Enantioselective Synthesis of Compounds of Formula Ib

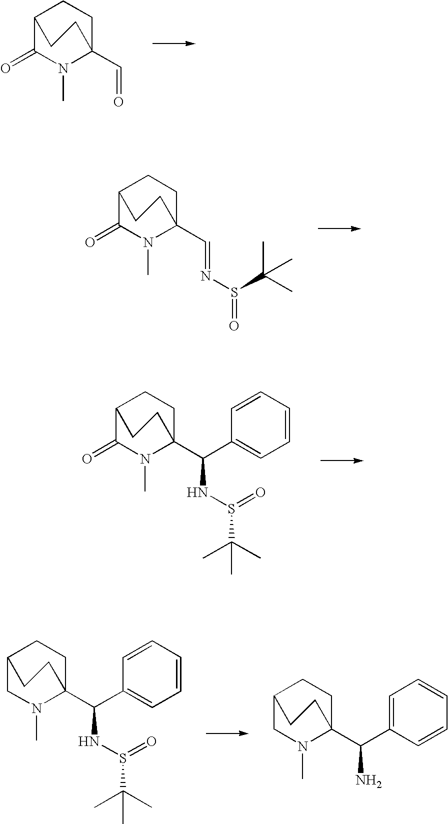

Method 4 depicts a generalized scheme suitable for enantioselective synthesis of compounds of Formula Ib. Those of skill in the art will readily recognize various reagents and intermediates or changes in moieties that could be used to make additional compounds of Formula Ib. R² and n can be selected as described elsewhere herein.

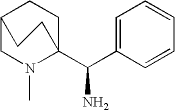

### EXAMPLE 5

### Preparation of (R)-(2-methyl-2-azabicyclo[2.2.2]octan-1-yl)(phenyl)methanamine

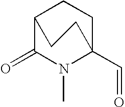

### Step A. Preparation of 2-methyl-3-oxo-2-azabicyclo[2.2.2]octane-l-carbaldehyde from methyl 2-methyl-3-oxo-2-azabicyclo[2.2.2]octane-1-carboxylate

To a cloudy solution of methyl 2-methyl-3-oxo-2-azabicyclo[2.2.2]octane-1-carboxylate (0.224 g, 1.14 mmol) from Example 1, Step A, in tetrahydrofuran (5.68 mL) at −78° C. was added 2.0 M lithium aluminum hydride in tetrahydrofuran (0.568 mL, 1.14 mmol) dropwise, maintaining the reaction temperature below −68° C. After 5 min, concentrated aqueous hydrochloric acid (0.095 mL, 1.14 mmol) was added dropwise, resulting in an exotherm, and the reaction temperature reached −38° C. before being cooled back down to −78° C. After 10 min, the reaction was warmed to −20° C., and then the white mixture was diluted with ethyl acetate and saturated aqueous sodium potassium tartrate (Rochelle's salt). The layers were separated, the aqueous layer was extracted with ethyl acetate (×2), and the combined organic layers were dried over sodium sulfate, filtered, and concentrated to afford crude 2-methyl-3-oxo-2-azabicyclo[2.2.2]octane-1-carbaldehyde (0.189 g, 100%) containing a small amount of alcohol (˜15%) as a clear colorless oil. 1H NMR (300 MHz, chloroform-d) δ ppm 1.65-1.96 (m, 8 H), 2.56-2.71 (m, 1 H), 3.02 (s, 3 H), 9.85 (s, 1 H). ESI⁺ LCMS (M+MeOH+H)⁺ 200.16.

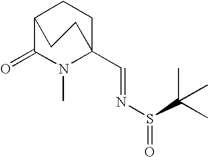

### Step B. Preparation of (R,E)-2-methyl-N-((2-methyl-3-oxo-2-azabicyclo[2.2.2]octan-1-yl)methylene)propane-2-sulfinamide from crude 2-methyl-3-oxo-2-azabicyclo[2.2.2]octane-1-carbaldehyde

To a solution of 2-methyl-3-oxo-2-azabicyclo[2.2.2]octane-1-carbaldehyde (0.726 g, 4.34 mmol) and tetraethoxytitanium (2.003 mL, 9.55 mmol) in tetrahydrofuran (10.85 mL) was added (R)-2-methylpropane-2-sulfinamide (0.632 g, 5.21 mmol). The resulting slightly cloudy white solution was stirred at ambient temperature for 15 h and then quenched with saturated aqueous sodium bicarbonate (10 drops). The resulting mixture was diluted with ethyl acetate (10 mL) and stirred vigorously for 30 min before being filtered through a pad of sodium sulfate. The filtrate was concentrated, and the resulting residue was purified by flash column chromatography (SiO₂, 0-100% ethyl acetate in dichloromethane) to afford (R,E)-2-methyl-N-((2-methyl-3-oxo-2-azabicyclo[2.2.2]octan-1-yl)methylene)propane-2-sulfinamide (0.553 g, 47%) as a clear oil which solidified to a white solid on standing. 1H NMR (500 MHz, chloroform-d) δ ppm 1.23 (s, 9H), 1.76-1.86 (m, 4H), 1.87-1.97 (m, 4H), 1.97-2.04 (m, 1H), 2.94 (s, 3H), 8.24 (s, 1 H). ESI⁺ LCMS (M+H)⁺ 271.2

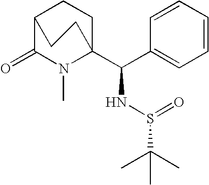

### Step C. Preparation of (R)-2-methyl-N-((R)-(2-methyl-3-oxo-2-azabicyclo[2.2.2]octan-1-yl)(phenyl)methyl)propane-2-sulfinamide from (R,E)-2-methyl-N-((2-methyl-3-oxo-2-azabicyclo[2.2.2]octan-1-yl)methylene)propane-2-sulfinamide

A solution of (R,E)-2-methyl-N-((2-methyl-3-oxo-2-azabicyclo[2.2.2]octan-1-yl)methylene)propane-2-sulfinamide (0.100 g, 0.37 mmol) in THF (2.0 mL) at −78° C. was treated with trimethylaluminum (2M in toluene, 0.200 mL, 0.40 mmol). Phenyllithium (1.8 M in di-n-butyl ether, 0.230 mL, 0.41 mmol) was added dropwise over 5 minutes. After 45 min, the reaction mixture was quenched with 1:1 saturated aqueous ammonium hydroxide and saturated aqueous ammonium chloride, the cooling bath was removed, and the mixture was warmed to ambient temperature. The mixture was then extracted with ethyl acetate (×2), and the combined organic layers were washed with water and saturated aqueous sodium chloride, dried over magnesium sulfate, filtered, and concentrated. The resulting solid was vacuum dried at ambient temperature for 20 min to afford (R)-2-methyl-N-((R)-(2-methyl-3-oxo-2-azabicyclo[2.2.2]octan-1-yl)(phenyl)methyl)propane-2-sulfinamide (0.140 g, 109%) containing a small amount of ethyl acetate. 1H NMR (300 MHz, chloroform-d) d ppm 1.02-1.20 (m, 1 H), 1.26 (s, 9 H), 1.42-2.05 (m, 7 H), 2.55-2.63 (m, 1 H), 3.20 (s, 3 H), 3.76 (s, 1 H), 4.80 (s, 1 H), 7.34 (s, 5 H). ESI⁺ LCMS (M+H)⁺ 349.3.

Stereochemical Determination: Reduction of the amide (See Step D below) and conversion of the resulting sulfinamide to the corresponding Boc carbamate (as described in Example 4) allowed for the determination that this compound was of 98% (R)-2-methyl-N-((R)-(2-methyl-3-oxo-2-azabicyclo[2.2.2]octan-1-yl)(phenyl)methyl)propane-2-sulfinamide using SFC conditions as described in Example 4, Step B. If the (S) enantiomer was desired, (S)-2-methylpropane-2-sulfinamide was used.

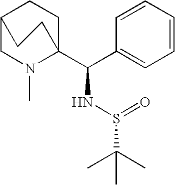

### Step D. Preparation of (R)-2-methyl-N-((R)-(2-methyl-2-azabicyclo[2.2.2]octan-1-yl)(phenyl)methyl)propane-2-sulfinamide from (R)-2-methyl-N-((R)-(2-methyl-3-oxo-2-azabicyclo[2.2.2]octan-1-yl)(phenyl)methyl)propane-2-sulfinamide

A solution of (R)-2-methyl-N-((R)-(2-methyl-2-azabicyclo[2.2.2]octan-1-yl)(phenyl)methyl)propane-2-sulfinamide (0.369 g, 1.06 mmol) in THF (6.0 mL) was treated with carbonylhydridotris(triphenylphosphine)rhodium(I) (0.030 g, 0.033 mmol) and diphenylsilane (0.500 mL, 2.69 mmol). After 1 hour, nitrogen was bubbled through the reaction mixture. Additional rhodium catalyst (0.010 g, 0.011 mmol) and diphenylsilane (0.250 mL, 1.35 mmol) were added, and the mixture stirred at ambient temperature for 16 h. The reaction was then diluted with ether and extracted with 1 N aqueous hydrogen chloride (×2). The organic layer was discarded, and the aqueous layers were combined and basified with saturated aqueous ammonium hydroxide. The aqueous layer was then extracted with ethyl acetate (×3), and the combined organic layers were dried over magnesium sulfate, filtered, and concentrated. The resulting residue was purified by flash column chromatography (SiO₂, 5% 2M ammonia in methanol in dichloromethane) to afford (R)-2-methyl-N-((R)-(2-methyl-2-azabicyclo[2.2.2]octan-1-yl)(phenyl)methyl)propane-2-sulfinamide (300mg, 83%) as a light yellow oil that solidified on standing. 1H NMR (300 MHz, chloroform-d) δ ppm 1.02-1.48 (m, 13 H), 1.48-2.00 (m, 5 H), 2.45 (s, 3 H), 2.48-2.63 (m, 1 H), 3.30 (dd, J=10.2, 1.6 Hz, 1 H), 4.35 (s, 1 H), 5.14 (s, 1 H), 7.18-7.37 (m, 5 H). ESI⁺ LCMS (M+H)⁺ 335.2140.

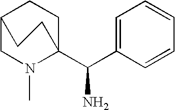

### Step E. Preparation of (R)-(2-methyl-2-azabicyclo[2.2.2]octan-1-yl)(phenyl)methanamine from (R)-2-methyl-N-((R)-(2-methyl-2-azabicyclo[2.2.2]octan-1-yl)(phenyl)methyl)propane-2-sulfinamide

The compound (R)-(2-methyl-2-azabicyclo[2.2.2]octan-1-yl)(phenyl)methanamine was prepared from (R)-2-methyl-N-((R)-(2-methyl-2-azabicyclo[2.2.2]octan-1-yl)(phenyl)methyl)propane-2-sulfinamide using the procedure of Example 1, Step F. This material could then be used as described in Example 4, Step D, to prepare single enantiomers of desired benzamides. 1H NMR (300 MHz, chloroform-d) δ ppm 0.95-1.14 (m, 1 H), 1.29-1.46 (m, 3 H), 1.48-1.71 (m, 5 H), 1.73-1.86 (m, 1 H), 1.97-2.08 (m, 1 H), 2.41-2.52 (m, 4 H), 3.28 (dt, J=10.6, 2.5 Hz, 1 H), 4.04 (s, 1 H), 7.16-7.39 (m, 5 H). ESI⁺ LCMS (M+H)⁺ 231.1847.

Exemplary compounds of Formula Ib which can be made by the processes described herein include:

| | | | | | Mass |
|---|---|---|---|---|---|
| | | | | | spectroscopy |
| | | | | | mass ion(s), |
| | | IC₅₀ | Synthesis | | (HPLC retention |
| structure | Isomer | (uM) | Method | Name | time, method) |
| 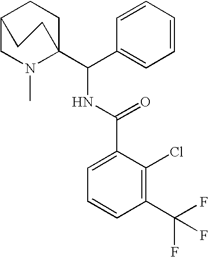 | RS | 0.0387 | 1 | 2-chloro-N-((2-methyl-2-azabicyclo[2.2.2]octan-1-yl)(phenyl)methyl)-3-(trifluoromethyl)benzamide | 437.09, 439.09(2.30 min; MS3) |
| 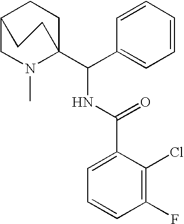 | RS | 0.012 | 1 | 2-chloro-3-fluoro-N-((2-methyl-2-azabicyclo[2.2.2]octan-1-yl)(phenyl)methyl)benzamide | 387.1637(0.89 min; MS2) |
| 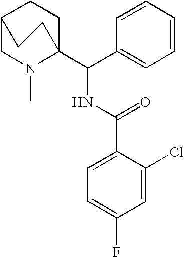 | RS | 0.00845 | 1 | 2-chloro-4-fluoro-N-((2-methyl-2-azabicyclo[2.2.2]octan-1-yl)(phenyl)methyl)benzamide | 387.1636(0.91 min; MS2) |
| 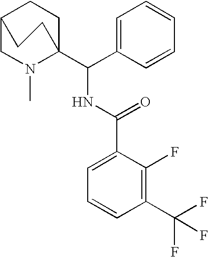 | RS | 0.092 | 1 | 2-fluoro-N-((2-methyl-2-azabicyclo[2.2.2]octan-1-yl)(phenyl)methyl)-3-(trifluoromethyl)benzamide | 421.1915(9.30 min; MS4) |
| 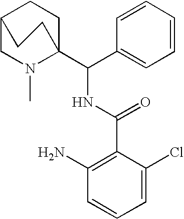 | RS | 0.00391 | 1 | 2-amino-6-chloro-N-((2-methyl-2-azabicyclo[2.2.2]octan-1-yl)(phenyl)methyl)benzamide | 384.1834(0.84 min; MS2) |
| 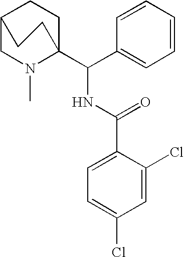 | RS | 0.00645 | 1 | 2,4-dichloro-N-((2-methyl-2-azabicyclo[2.2.2]octan-1-yl)(phenyl)methyl)benzamide | 403.1329, 405.1308(0.95 min; MS2) |
| 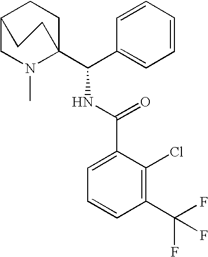 | S | 0.0285 | 2 | (S)-2-chloro-N-((2-methyl-2-azabicyclo[2.2.2]octan-1-yl)(phenyl)methyl)-3-(trifluoromethyl)benzamide | 437.2, 439.2(0.63 min; MS1) |
| 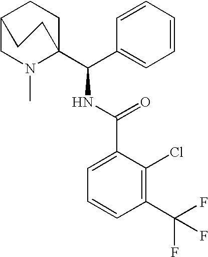 | R | 0.00629 | 2, 3 | (R)-2-chloro-N-((2-methyl-2-azabicyclo[2.2.2]octan-1-yl)(phenyl)methyl)-3-(trifluoromethyl)benzamide,citric acid salt | 437.2, 439.2(0.63 min; MS1) |
| 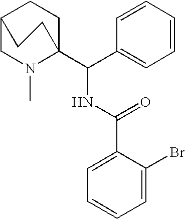 | RS | 0.0105 | 1 | 2-bromo-N-((2-methyl-2-azabicyclo[2.2.2]octan-1-yl)(phenyl)methyl)benzamide | 413.1217, 415.1210(0.90 min; MS2) |
| 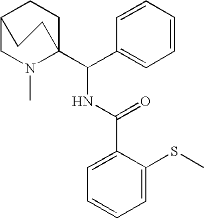 | RS | 0.00295 | 1 | N-((2-methyl-2-azabicyclo[2.2.2]octan-1-yl)(phenyl)methyl)-2-(methylthio)benzamide | 381.1988(0.91 min; MS2) |
| 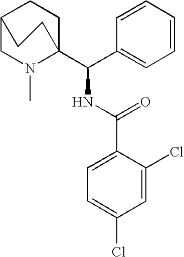 | R | 0.0111 | 2, 3 | (R)-2,4-dichloro-N-((2-methyl-2-azabicyclo[2.2.2]octan-1-yl)(phenyl)methyl)benzamide,citric acid salt | 403.1340, 405.1315(0.99 min; MS2) |
| 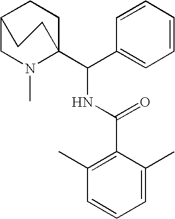 | RS | <0.000483 | 1 | 2,6-dimethyl-N-((2-methyl-2-azabicyclo[2.2.2]octan-1-yl)(phenyl)methyl)benzamide | 363.243(0.92 min; MS2) |
| 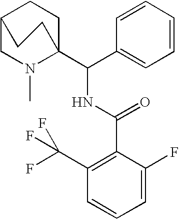 | RS | 0.065 | 1 | 2-fluoro-N-((2-methyl-2-azabicyclo[2.2.2]octan-1-yl)(phenyl)methyl)-6-(trifluoromethyl)benzamide | 421.1891(0.94 min; MS2) |
| 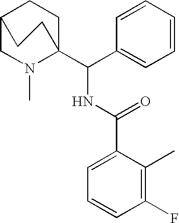 | RS | 0.0026 | 1 | 3-fluoro-2-methyl-N-((2-methyl-2-azabicyclo[2.2.2]octan-1-yl)(phenyl)methyl)benzamide | 367.218(0.94 min; MS2) |
| 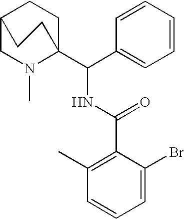 | RS | 0.00134 | 3 | 2-bromo-6-methyl-N-((2-methyl-2-azabicyclo[2.2.2]octan-1-yl)(phenyl)methyl)benzamide | 427.1359, 429.1342(0.93 min; MS2) |
| 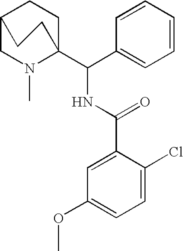 | RS | 0.0327 | 1 | 2-chloro-5-methoxy-N-((2-methyl-2-azabicyclo[2.2.2]octan-1-yl)(phenyl)methyl)benzamide | 399.1811(0.94 min; MS2) |
| 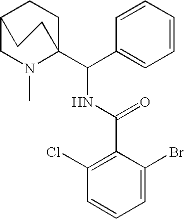 | RS | 0.0121 | 1 | 2-bromo-6-chloro-N-((2-methyl-2-azabicyclo[2.2.2]octan-1-yl)(phenyl)methyl)benzamide | 447.0827, 449.0801(0.93 min; MS2) |
| 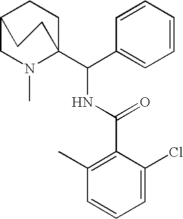 | RS | 0.0023 | 1 | 2-chloro-6-methyl-N-((2-methyl-2-azabicyclo[2.2.2]octan-1-yl)(phenyl)methyl)benzamide | 383.1865(0.91 min; MS2) |
| 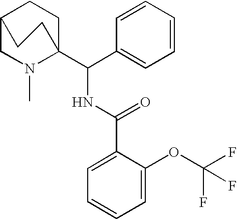 | RS | 0.0136 | 1 | N-((2-methyl-2-azabicyclo[2.2.2]octan-1-yl)(phenyl)methyl)-2-(trifluoromethoxy)benzamide | 419.1938(1.00 min; MS2) |
| 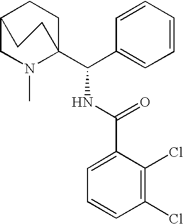 | S | 0.205 | 3 | (S)-2,3-dichloro-N-((2-methyl-2-azabicyclo[2.2.2]octan-1-yl)(phenyl)methyl)benzamide,citric acid salt | 403.1325, 405.1299(0.98 min; MS2) |
| 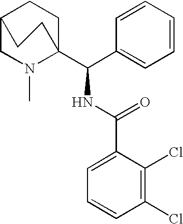 | R | 0.0011 | 3 | (R)-2,3-dichloro-N-((2-methyl-2-azabicyclo[2.2.2]octan-1-yl)(phenyl)methyl)benzamide,citric acid salt | 403.1333, 405.1315(0.98 min; MS2) |
| 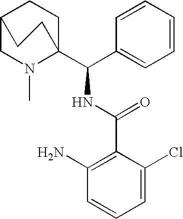 | R | 0.00772 | 3 | (R)-2-amino-6-chloro-N-((2-methyl-2-azabicyclo[2.2.2]octan-1-yl)(phenyl)methyl)benzamide,citric acid salt | 384.1838(0.88 min; MS2) |
| 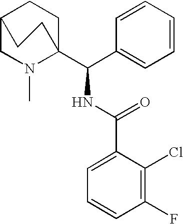 | R | 0.0059 | 3 | (R)-2-chloro-3-fluoro-N-((2-methyl-2-azabicyclo[2.2.2]octan-1-yl)(phenyl)methyl)benzamide,citric acid salt | 387.1639(0.92 min; MS2) |
| 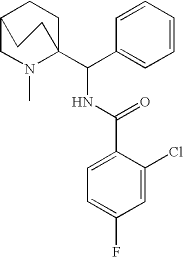 | R | 0.00771 | 3 | (R)-2-chloro-4-fluoro-N-((2-methyl-2-azabicyclo[2.2.2]octan-1-yl)(phenyl)methyl)benzamide,citric acid salt | 387.1639(0.92 min; MS2) |
| 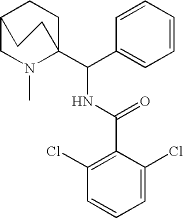 | RS | 0.046 | 1 | 2,6-dichloro-N-((2-methyl-2-azabicyclo[2.2.2]octan-1-yl)(phenyl)methyl)benzamide | 403.1332, 405.1306(0.92 min; MS2) |
| 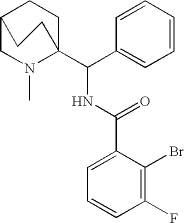 | RS | 0.0258 | 1 | 2-bromo-3-fluoro-N-((2-methyl-2-azabicyclo[2.2.2]octan-1-yl)(phenyl)methyl)benzamide | 431.1111, 433.1100(0.92 min; MS2) |
| 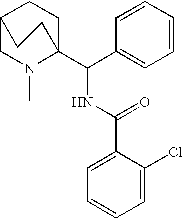 | RS | 0.0651 | 1 | 2-chloro-N-((2-methyl-2-azabicyclo[2.2.2]octan-1-yl)(phenyl)methyl)benzamide | 369.1728(0.88 min; MS2) |
| 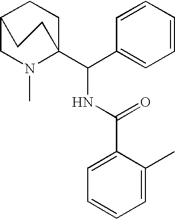 | RS | 0.00348 | 1 | 2-methyl-N-((2-methyl-2-azabicyclo[2.2.2]octan-1-yl)(phenyl)methyl)benzamide | 349.227(0.89 min; MS2) |
| 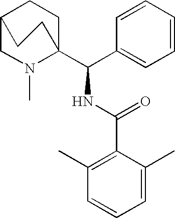 | R | 0.00127 | 3 | (R)-2,6-dimethyl-N-((2-methyl-2-azabicyclo[2.2.2]octan-1-yl)(phenyl)methyl)benzamide,citric acid salt | 363.2425(0.91 min; MS2) |
| 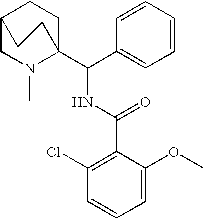 | RS | 0.00315 | 1 | 2-chloro-6-methoxy-N-((2-methyl-2-azabicyclo[2.2.2]octan-1-yl)(phenyl)methyl)benzamide | 399.1835(0.89 min; MS2) |
| 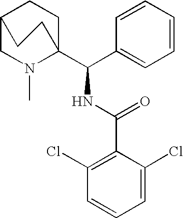 | R | 0.0233 | 3 | (R)-2,6-dichloro-N-((2-methyl-2-azabicyclo[2.2.2]octan-1-yl)(phenyl)methyl)benzamide,citric acid salt | 403.2, 405.2,407.2 (0.52 min;MS1) |
| 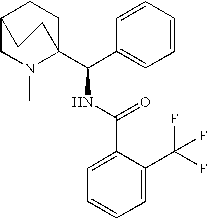 | R | 0.0496 | 3 | (R)-N-((2-methyl-2-azabicyclo[2.2.2]octan-1-yl)(phenyl)methyl)-2-(trifluoromethyl)benzamide,citric acid salt | 403.1978(0.96 min; MS2) |
| 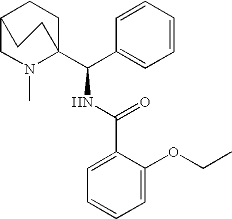 | R | 0.336 | 3 | (R)-2-ethoxy-N-((2-methyl-2-azabicyclo[2.2.2]octan-1-yl)(phenyl)methyl)benzamide,citric acid salt | 379.2381(1.01 min; MS2) |
| 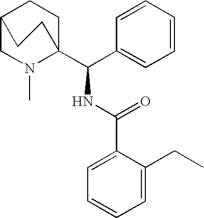 | R | 0.000965 | 3 | (R)-2-ethyl-N-((2-methyl-2-azabicyclo[2.2.2]octan-1-yl)(phenyl)methyl)benzamide,citric acid salt | 363.2426(0.98 min; MS2) |
| 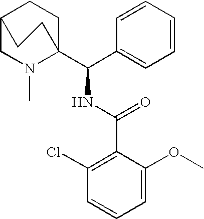 | R | 0.00474 | 3 | (R)-2-chloro-6-methoxy-N-((2-methyl-2-azabicyclo[2.2.2]octan-1-yl)(phenyl)methyl)benzamide,citric acid salt | 399.1817(0.91 min; MS2) |
| 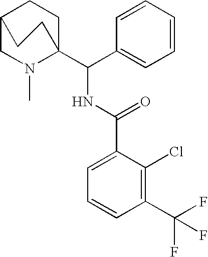 | RS | | | 2-chloro-N-((2-methyl-2-azabicyclo[2.2.2]octan-1-yl)(phenyl)methyl)-3-(trifluoromethyl)benzamide | |
| 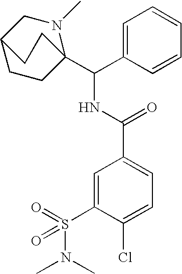 | RS | | | 4-chloro-3-(N,N-dimethylsulfamoyl)-N-((2-methyl-2-azabicyclo[2.2.2]octan-1-yl)(phenyl)methyl)benzamide | |
| 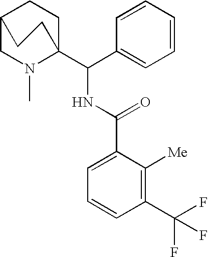 | RS | <0.00234 | 1 | 2-methyl-N-((2-methyl-2-azabicyclo[2.2.2]octan-1-yl)(phenyl)methyl)-3-(trifluoromethyl)benzamide | 417.2154(1.00 min; MS2) |
| 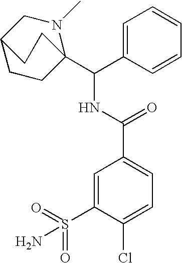 | RS | | | 4-chloro-N-((2-methyl-2-azabicyclo[2.2.2]octan-1-yl)(phenyl)methyl)-3-sulfamoylbenzamide | |
| 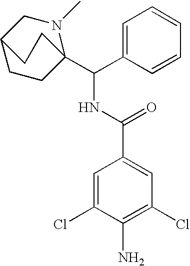 | RS | | | 4-amino-3,5-dichloro-N-((2-methyl-2-azabicyclo[2.2.2]octan-1-yl)(phenyl)methyl)benzamide | |
| 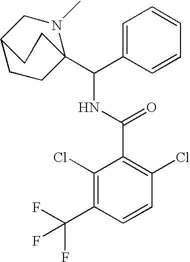 | RS | | | 2,6-dichloro-N-((2-methyl-2-azabicyclo[2.2.2]octan-1-yl)(phenyl)methyl)-3-(trifluoromethyl)benzamide | |
| 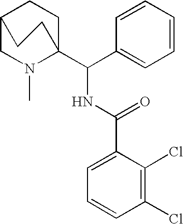 | RS | <0.0025 | 1 | 2,3-dichloro-N-((2-methyl-2-azabicyclo[2.2.2]octan-1-yl)(phenyl)methyl)benzamide | 403.1345, 405.1324(0.96 min; MS2) |
| 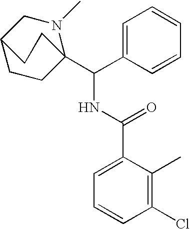 | RS | | | 3-chloro-2-methyl-N-((2-methyl-2-azabicyclo[2.2.2]octan-1-yl)(phenyl)methyl)benzamide | |
| 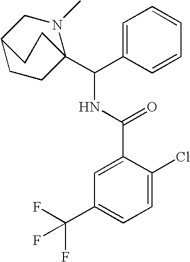 | RS | | | 2-chloro-N-((2-methyl-2-azabicyclo[2.2.2]octan-1-yl)(phenyl)methyl)-5-(trifluoromethyl)benzamide | |
| 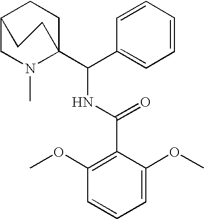 | RS | 0.00581 | 1 | 2,6-dimethoxy-N-((2-methyl-2-azabicyclo[2.2.2]octan-1-yl)(phenyl)methyl)benzamide | 395.2319(0.88 min; MS2) |
| 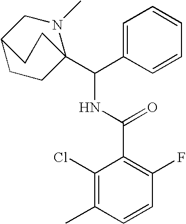 | RS | | | 2-chloro-6-fluoro-3-methyl-N-((2-methyl-2-azabicyclo[2.2.2]octan-1-yl)(phenyl)methyl)benzamide | |
| 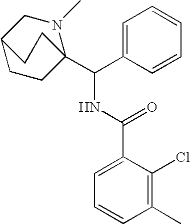 | RS | | | 2-chloro-3-methyl-N-((2-methyl-2-azabicyclo[2.2.2]octan-1-yl)(phenyl)methyl)benzamide | |
| 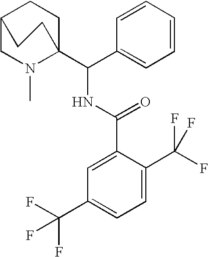 | RS | 0.0862 | 1 | N-((2-methyl-2-azabicyclo[2.2.2]octan-1-yl)(phenyl)methyl)-2,5-bis(trifluoromethyl)benzamide | 471.1862(1.10 min; MS2) |
| 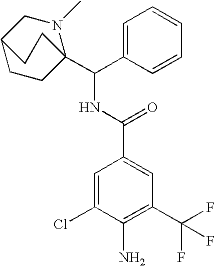 | RS | | | 4-amino-3-chloro-N-((2-methyl-2-azabicyclo[2.2.2]octan-1-yl)(phenyl)methyl)-5-(trifluoromethyl)benzamide | |
| 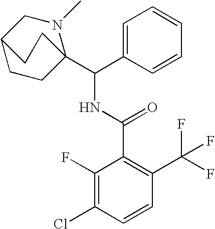 | RS | | | 3-chloro-2-fluoro-N-((2-methyl-2-azabicyclo[2.2.2]octan-1-yl)(phenyl)methyl)-6-(trifluoromethyl)benzamide | |
| 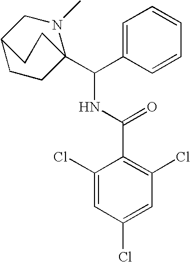 | RS | | | 2,4,6-trichloro-N-((2-methyl-2-azabicyclo[2.2.2]octan-1-yl)(phenyl)methyl)benzamide | |

### Method 5. Racemic Synthesis of Compounds of Formula Ia.

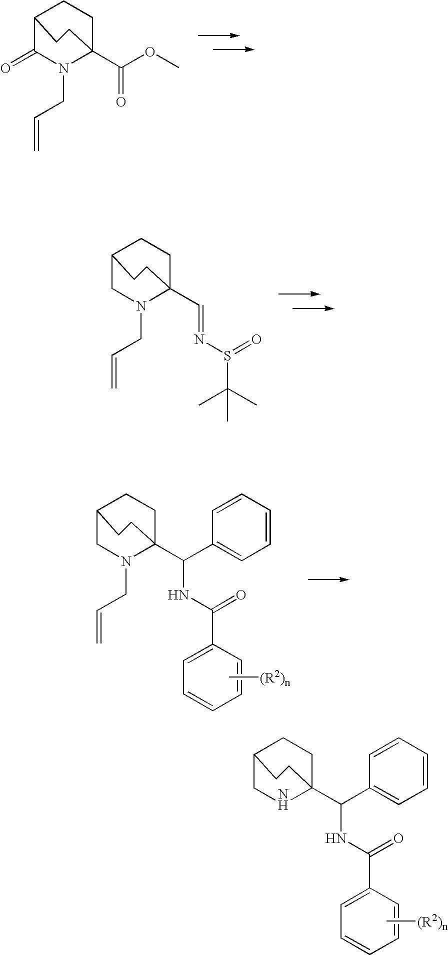

Method 5 depicts a generalized scheme suitable for racemic synthesis of compounds of Formula la. Those of skill in the art will readily recognize various reagents and intermediates or changes in moieties that could be used to make additional compounds of Formula la. R² and n can be selected as described elsewhere herein.

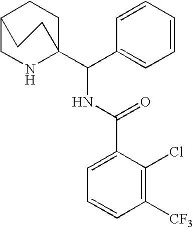

### EXAMPLE 6

### Preparation of N-(2-azabicyclo[2.2.2]octan-1-yl(phenyl)methyl)-2-chloro-3-(trifluoromethyl)benzamide

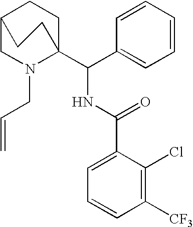

### Step A. Preparation of N-((2-allyl-2-azabicyclo[2.2.2]octan-1-yl)(phenyl)methyl)-2-chloro-3-(trifluoromethyl)benzamide from (2-allyl-2-azabicyclo[2.2.2]octan-1-yl)(phenyl)methanamine hydrochloride

To a mixture of 2-chloro-3-(trifluoromethyl)benzoic acid (0.217 g, 0.97 mmol) in dichloromethane (6.90 mL) was added oxalyl chloride (0.121 mL, 1.38 mmol) and one drop of N,N-dimethylformamide. The resulting mixture was stirred at room temperature for 30 min, whereupon it became a clear solution and was concentrated. The resulting light yellow oil was redissolved in dichloromethane (5 mL).

To a mixture of (2-allyl-2-azabicyclo[2.2.2]octan-1-yl)(phenyl)methanamine hydrochloride (0.202 g, 0.69 mmol; prepared according to the procedures of Example 1, Steps A-F, substituting allyl iodide for iodomethane in step A, and forgoing basification in Step F), triethylamine (0.385 mL, 2.76 mmol), and dichloromethane (6.90 mL) was added via cannula the acid chloride prepared above. The resulting light orange mixture was stirred at room temperature for 1 h and then quenched with saturated aqueous sodium bicarbonate and extracted with dichloromethane (×1) and ethyl acetate (×1). The resulting organic layers were dried over sodium sulfate, filtered and concentrated. The resulting residue was purified by flash column chromatography (SiO₂, 0-50% ethyl acetate in hexanes) to afford partially pure product N-((2-allyl-2-azabicyclo[2.2.2]octan-1-yl)(phenyl)methyl)-2-chloro-3-(trifluoromethyl)benzamide (0.215 g, 67.3%) as a faint yellow foam solid. 1H NMR (500 MHz, DMSO-d₆) δ ppm 1.36 (d, J=5.1 Hz, 4 H), 1.46-1.61 (m, 3 H), 1.71-1.80 (m, 1 H), 1.95-2.05 (m, 1 H), 2.55 (d, J=11.1 Hz, 1 H), 2.89 (d, J=11.1 Hz, 1 H), 3.21 (dd, J=13.8, 6.9 Hz, 1 H), 3.46-3.53 (m, 1 H), 5.11 (d, 1 H), 5.20 (d, 1 H), 5.25 (dd, J=17.2, 1.4 Hz, 1 H), 5.75-5.91 (m, 1 H), 7.20-7.26 (m, 1 H), 7.28-7.35 (m, 4 H), 7.52-7.58 (m, 1 H), 7.62 (t, J=7.7 Hz, 1 H), 7.91 (dd, J=7.8, 1.3 Hz, 1 H), 8.91 (d, J=8.4 Hz, 1 H). ESI⁺ LCMS (M+H)⁺ 463.2, 465.2.

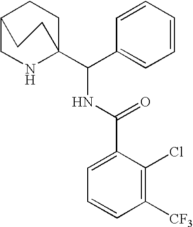

### Step B. Preparation of N-(2-azabicyclo[2.2.2]octan-1-yl(phenyl)methyl)-2-chloro-3-(trifluoromethyl)benzamide from N-((2-allyl-2-azabicyclo[2.2.2]octan-1-yl)(phenyl)methyl)-2-chloro-3-(trifluoromethyl)benzamide

To a degassed solution of tetrakis(triphenylphosphine)palladium(0) (4.74 mg, 4.10 μmol) and 1,3-dimethylbarbituric acid (0.192 g, 1.23 mmol) in dichloromethane (3 mL) at 30° C. was added a solution of N-((2-allyl-2-azabicyclo[2.2.2]octan-1-yl)(phenyl)methyl)-2-chloro-3-(trifluoromethyl)benzamide (0.190 g, 0.41 mmol) in 3 mL of dichloromethane, resulting in a light orange-yellow solution. This solution was maintained at 30° C. with stirring for 60 min. The orange solution was cooled to room temperature and quenched with saturated aqueous sodium hydrogen sulfate. The mixture was then extracted with dichloromethane (×2) and ethyl acetate (×1). The combined organic layers were dried over sodium sulfate, filtered, and concentrated. The resulting residue was partially purified by flash column chromatography (SiO₂, 0-5% methanolic ammonia in ethyl acetate) to afford N-(2-azabicyclo[2.2.2]octan-1-yl(phenyl)methyl)-2-chloro-3-(trifluoromethyl)benzamide as an orange solid. This material was further purified by preparative LCMS (C18, acetonitrile in water containing ammonium carbonate, pH 10) to afford N-(2-azabicyclo[2.2.2]octan-1-yl (phenyl)methyl)-2-chloro-3-(trifluoromethyl)benzamide (76 mg, 44%) of 95% purity as a faint pink solid. 1H NMR (300 MHz, DMSO-d₆) δ ppm 1.39-1.82 (m, 10 H), 2.75-2.86 (m, 2 H), 4.81 (d, J=9.1 Hz, 1 H), 7.19-7.37 (m, 5 H), 7.56-7.63 (m, 2 H), 7.85-7.96 (m, 1 H), 8.86 (d, J=9.1 Hz, 1 H). ESI⁺ LCMS (M+H)⁺ 423.14, 425.2.

### Method 6. Preparation of Chiral Compounds of Formula Ia by Resolution of an Intermediate

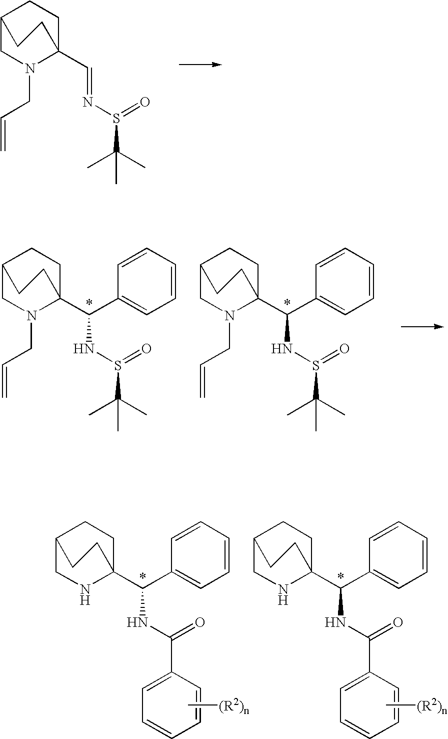

Method 6 depicts a generalized scheme suitable for preparation of chiral compounds of Formula Ia by resolution of an intermediate. Those of skill in the art will readily recognize various reagents and intermediates or changes in moieties that could be used to make additional compounds of Formula la. R² and n can be selected as described elsewhere herein.

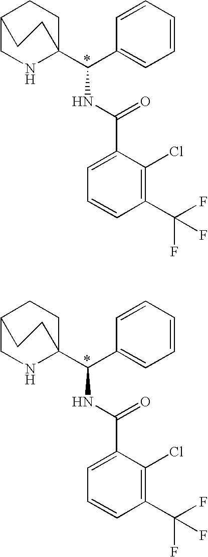

### EXAMPLES 7 AND 8

### Preparation of (S*)-N-(2-azabicyclo[2.2.2]octan-1-yl(phenyl)methyl)-2-chloro-3-(trifluoromethyl)benzamide and (R*)-N-(2-azabicyclo[2.2.2]octan-1-yl(phenyl)methyl)-2-chloro-3-(trifluoromethyl)benzamide

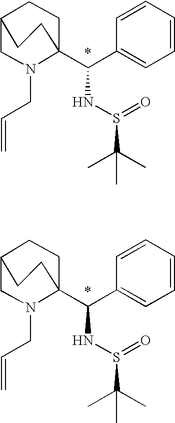

### Step A. Preparation of (S)-N-((S*)-(2-allyl-2-azabicyclo[2.2.2]octan-1-yl)(phenyl)methyl)-2-methylpropane-2-sulfinamide and (S)-N-((R*)-(2-allyl-2-azabicyclo[2.2.2]octan-1-yl)(phenyl)methyl)-2-methylpropane-2-sulfinamide from (S,E)-N-((2-allyl-2-azabicyclo[2.2.2]octan-1-yl)methylene)-2-methylpropane-2-sulfinamide

To a solution of (S,E)-N-((2-allyl-2-azabicyclo[2.2.2]octan-1-yl)methylene)-2-methylpropane-2-sulfinamide (1.0 g, 3.54 mmol; prepared according to the procedures of Example 1, Steps A-E, substituting allyliodide for iodomethane in Step A and (S)-2-methylpropane-2-sulfinamide for 2-methylpropane-2-sulfonamide in Step D) and tetrahydrofuran (7.08 ml) was added 1.0 M phenylmagnesium bromide in tetrahydrofuran (10.62 ml, 10.62 mmol), affording an orange solution. After 30 min, the reaction became red. After 2 h and 4h, another 1 mL of 1.0 M phenylmagnesium bromide in tetrahydrofuran was added. The resulting orange solution was stirred at room temperature for 16 h and then another 2 mL of 1.0 M phenylmagnesium bromide in tetrahydrofuran were added. The orange solution was stirred for 2.5 days at ambient temperature and then quenched with 50% saturated aqueous ammonium chloride and saturated aqueous ammonium hydroxide. The mixture was diluted with ethyl acetate and stirred for 15 min. The layers were then separated and the aqueous layer was extracted with ethyl acetate (×3). The combined organic layers were dried over sodium sulfate, filtered and concentrated to a yellow oil. This oil was purified by preparative HPLC (C18, 30-90% acetonitrile in water containing ammonium bicarbonate, pH 10). The faster product peak was further repurified by flash column chromatography (SiO₂, 0-100% ethyl aceate in hexanes, then 10% methanol in ethyl acetate after 5 min to afford the arbitrarily assigned (S)-N-((R*)-(2-allyl-2-azabicyclo[2.2.2]octan-1-yl)(phenyl)methyl)-2-methylpropane-2-sulfinamide (0.335 g, 26.2%) of 95% purity as a white solid. The second diastereomer was isolated from HPLC fractions and arbitrarily assigned as (S)-N-((S*)-(2-allyl-2-azabicyclo[2.2.2]octan-1-yl)(phenyl)methyl)-2-methylpropane-2-sulfinamide (0.495 g, 38.8%) of 93% purity as a viscous light yellow oil. (S)-N-((R*)-(2-allyl-2-azabicyclo[2.2.2]octan-1-yl)(phenyl)methyl)-2-methylpropane-2-sulfinamide: 1H NMR (300 MHz, chloroform-d) δ ppm 1.10 (s, 9 H), 1.12-1.51 (m, 4 H), 1.52-1.68 (m, 4 H), 1.72-2.00 (m, 2 H), 2.65 (dt, J=11.7, 2.8 Hz, 1 H), 3.02 (t, J=7.2 Hz, 1 H), 3.56-3.75 (m, 1 H), 4.40 (d, J=0.8 Hz, 1 H), 5.09 (s, 1 H), 5.13-5.22 (m, 1 H), 5.23-5.35 (m, 1 H), 5.74-5.93 (m, 1 H), 7.21-7.45 (m, 5 H). ESI⁺LCMS (M+H)⁺ 361.1. (S)-N-((S*)-(2-allyl-2-azabicyclo[2.2.2]octan-1-yl)(phenyl)methyl)-2-methylpropane-2-sulfinamide: 1H NMR (300 MHz, chloroform-d) δ ppm 1.20-1.37 (m, 2 H), 1.26 (s, 9 H), 1.37-1.51 (m, 2 H), 1.50-1.69 (m, 3 H), 1.69-1.95 (m, 2 H), 2.69 (dt, J=11.6, 2.9 Hz, 1 H), 2.89 (dd, J=13.7, 7.4 Hz, 1 H), 3.06-3.16 (m, 1 H), 3.74-3.85 (m, 1 H), 4.48 (s, 1 H), 5.09-5.16 (m, 1 H), 5.21 (s, 1 H), 5.22-5.32 (m, 1 H), 5.75-5.96 (m, 1 H), 7.26-7.32 (m, 5 H). ESI⁺ LCMS (M+H)⁺ 361.5.

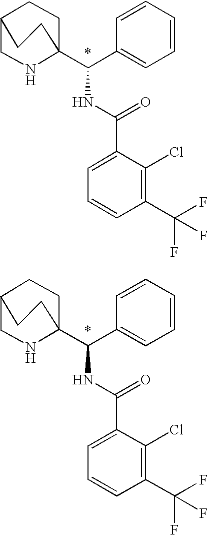

### Step B. Preparation of (S*)-N-(2-azabicyclo[2.2.2]octan-1-yl(phenyl)methyl)-2-chloro-3-(trifluoromethyl)benzamide and (R*)-N-(2-azabicyclo[2.2.2]octan-1-yl(phenyl)methyl)-2-chloro-3-(trifluoromethyl)benzamide from (S)-N-((S*)-(2-allyl-2-azabicyclo[2.2.2]octan-1-yl)(phenyl)methyl)-2-methylpropane-2-sulfinamide and (S)-N-((R*)-(2-allyl-2-azabicyclo[2.2.2]octan-1-yl)(phenyl)methyl)-2-methylpropane-2-sulfinamide

(S*)-N-(2-azabicyclo[2.2.2]octan-1-yl(phenyl)methyl)-2-chloro-3-(trifluoromethyl)benzamide and (R*)-N-(2-azabicyclo[2.2.2]octan-1-yl (phenyl)methyl)-2-chloro-3-(trifluoromethyl)benzamide were prepared according to the procedures of Example 1, Steps F (deprotection); Example 1, Step G (amide coupling) substituting 2-chloro-3-trifluoromethylbenzoic acid for 2,4-dichlorobenzoic acid and purifying final products via preparative HPLC (C18, acetonitrile in water containing ammonium carbonate, pH 10) rather than flash column chromatography; and Example 6, Step B (allyl deprotection) using a 20 min reaction time and purifying compounds via preparative HPLC (C18, acetonitrile in water containing ammonium carbonate, pH 10) rather than preparative LCMS. (S*)-N-(2-azabicyclo[2.2.2]octan-1-yl(phenyl)methyl)-2-chloro-3-(trifluoromethyl)benzamide: 1H NMR (500 MHz, chloroform-d) δ ppm 1.28-1.37 (m, 1 H), 1.55-1.71 (m, 7 H), 1.71-1.80 (m, 1 H), 2.06-2.16 (m, 1 H), 2.85-2.94 (m, 2 H), 4.83 (d, J=8.2 Hz, 1 H), 7.26-7.31 (m, 3 H), 7.31-7.37 (m, 2 H), 7.41 (t, J=7.7 Hz, 1 H), 7.46 (br. s, 1 H), 7.46 (d, J=7.6 Hz, 1 H), 7.75 (d, J=7.9 Hz, 1 H). ESI⁺ LCMS (M+H)⁺ 423.2, 425.2. (R*)-N-(2-azabicyclo[2.2.2]octan-1-yl(phenyl)methyl)-2-chloro-3-(trifluoromethyl)benzamide: 1H NMR (500 MHz, chloroform-d) δ ppm 1.29-1.37 (m, 1 H), 1.48-1.56 (m, 1 H), 1.56-1.70 (m, 6 H), 1.72-1.80 (m, 1 H), 2.05-2.18 (m, 1 H), 2.85-2.96 (m, 2 H), 4.83 (d, J=7.9 Hz, 1 H), 7.25-7.31 (m, 3 H), 7.32-7.37 (m, 2 H), 7.41 (t, J=7.8 Hz, 1 H), 7.48 (d, J=6.4 Hz, 1 H), 7.60-7.65 (m, 1 H), 7.75 (d, J=7.9 Hz, 1 H). ESI⁺ LCMS (M+H)⁺ 423.1445, 425.1421.

### Method 7. Enantioselective Synthesis of Compounds of Formula Ia.

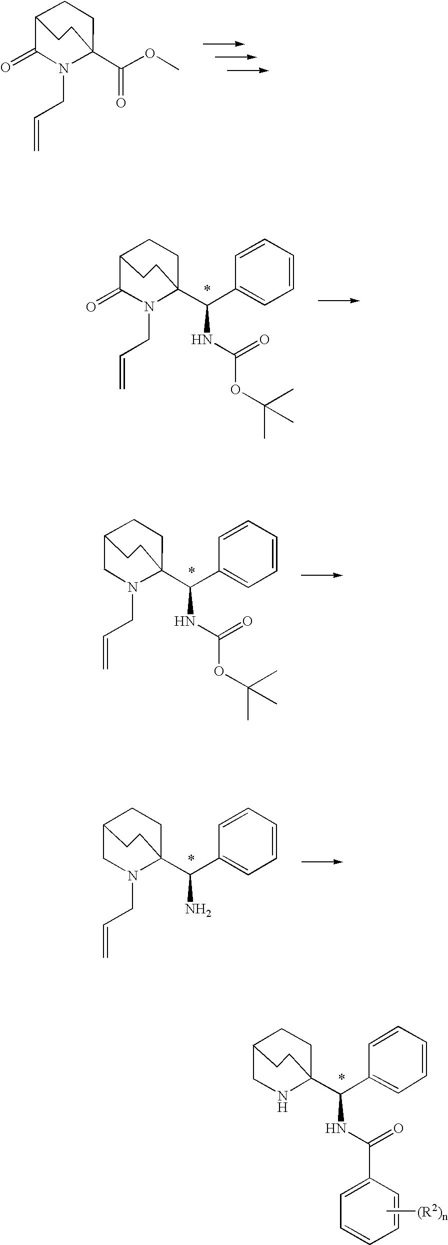

Method 7 depicts a generalized scheme suitable for enantioselective synthesis of compounds of Formula Ia. Those of skill in the art will readily recognize various reagents and intermediates or changes in moieties that could be used to make additional compounds of Formula Ia. R² and n can be selected as described elsewhere herein.

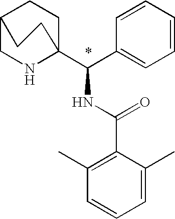

### EXAMPLE 9

### Preparation of (R*)-N-(2-azabicyclo[2.2.2]octan-1-yl(phenyl)methyl)-2,6-dimethylbenzamide

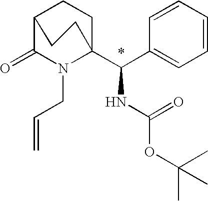

### Step A. Preparation of (R*)-tert-butyl(2-allyl-3-oxo-2-azabicyclo[2.2.2]octan-1-yl)(phenyl)methylcarbamate from (R)-N-((R)-(2-allyl-2-azabicyclo[2.2.2]octan-1-yl)(phenyl)methyl)-2-methylpropane-2-sulfinamide

To a solution of (R*)-N-((R)-(2-allyl-2-azabicyclo[2.2.2]octan-1-yl)(phenyl)methyl)-2-methylpropane-2-sulfinamide (0.654 g, 1.75 mmol; prepared according to the procedures of Example 5, Steps A-C, substituting methyl 2-allyl-3-oxo-2-aza-bicyclo[2.2.2]octane-1-carboxylate for methyl 2-methyl-3-oxo-2-azabicyclo[2.2.2]octane-1-carboxylate in Step A) in methanol (3 mL) was added 4M hydrochloric acid in dioxane (2.0 mL, 8.00 mmol). After 1 min, the reaction was concentrated to a white foamy solid. This solid was treated with 10 mL of saturated aqueous sodium bicarbonate and ethyl acetate (10 mL). To this mixture was added di-tert-butyl dicarbonate (0.973 mL, 4.19 mmol), and the resulting reaction was stirred vigorously for 16 h. This mixture was then extracted with ethyl acetate (×3), and the combined organic layers were dried over sodium sulfate, filtered, and concentrated. The resulting residue was purified by flash column chromatography (SiO₂, 0-100% ethyl acetate in hexanes) to afford (R*)-tert-butyl (2-allyl-3-oxo-2-azabicyclo[2.2.2]octan-1-yl)(phenyl)methylcarbamate of approximately 96% ee (Chiralpak IA column, SFC conditions using 15% methanol containing 0.3% isopropylamine). This material was further purified under supercritical fluid chromatography (liquid CO₂) employing isocratic 15% methanol containing 0.5% dimethylethylamine to afford enantiopure (R*)-tert-butyl(2-allyl-3-oxo-2-azabicyclo[2.2.2]octan-1-yl)(phenyl)methylcarbamate (0.578 g, 89%) as a white foam solid. The opposite enantiomer could be obtained using (S)-2-methylpropane-2-sulfinamide. 1H NMR (300 MHz, chloroform-d) δ ppm 1.06-1.20 (m, 1 H), 1.36 (br. s., 9 H), 1.55-1.93 (m, 7 H), 2.62 (quin, J=2.7 Hz, 1 H), 3.93 (dd, J=16.2, 7.2 Hz, 1 H), 4.58 (d, J=15.0 Hz, 1 H), 5.00-5.15 (m, 1 H), 5.21 (dd, J=10.3, 1.3 Hz, 1 H), 5.26-5.40 (m, 2 H), 5.85-6.02 (m, 1 H), 7.18-7.24 (m, 2 H), 7.26-7.37 (m, 3 H). ESI⁺ 371.3.

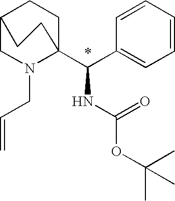

### Step B. Preparation of (R*)-tert-butyl(2-allyl-2-azabicyclo[2.2.2]octan-1-yl)(phenyl)methylcarbamate from (R*)-tert-butyl(2-allyl-3-oxo-2-azabicyclo[2.2.2]octan-1-yl)(phenyl)methylcarbamate

To a solution of sulfuric acid (0.123 mL, 2.31 mmol) in tetrahydrofuran (6 mL) at 0° C. was added 2.0 M lithium aluminum hydride in tetrahydrofuran (2.312 mL, 4.62 mmol) slowly. After 15 min, to the white mixture was added (R*)-tert-butyl(2-allyl-3-oxo-2-azabicyclo[2.2.2]octan-1-yl)(phenyl)methylcarbamate (0.571 g, 1.54 mmol) as a solution in tetrahydrofuran (2 mL) followed by a 2 mL wash. The reaction was stirred for 10 min, then quenched at 0° C. with sodium sulfate decahydrate and diluted with ethyl acetate. After 1 min, the white mixture was filtered, and the filtrate was concentrated to afford crude (R*)-tert-butyl(2-allyl-2-azabicyclo[2.2.2]octan-1-yl)(phenyl)methylcarbamate (0.498 g, 91 %) as an oily white solid. 1H NMR (300 MHz, chloroform-d) δ ppm 1.17-1.73 (m, 16 H), 1.77-1.92 (m, 1 H), 2.51-2.67 (m, 1 H), 2.92 (dd, J=13.7, 7.4 Hz, 1 H), 3.10 (d, J=11.2 Hz, 1 H), 3.44-3.59 (m, 1 H), 4.48 (br. s., 1 H), 5.11 (d, J=10.1 Hz, 1 H), 5.24 (dd, J=17.1, 1.3 Hz, 1 H), 5.72-5.99 (m, 2 H), 7.16-7.32 (m, 6 H). ESI⁺ LCMS (M+H)⁺ 357.3.

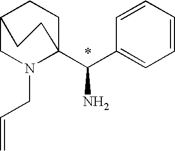

### Step C. Preparation of (R*)-(2-allyl-2-azabicyclo[2.2.2]octan-1-yl)(phenyl)methanamine from (R*)-tert-butyl(2-allyl-2-azabicyclo[2.2.2]octan-1-yl)(phenyl)methylcarbamate

To tert-butyl(2-allyl-2-azabicyclo[2.2.2]octan-1-yl)(phenyl)methylcarbamate (0.2 g, 0.56 mmol) was added 12N aqueous hydrochloric acid (1.0 mL, 12.00 mmol). After gas evolution ceased (1.0 min), the resulting cloudy solution was concentrated to a glass. This glass was reconcentrated from 10% methanol in dichloromethane, treated with saturated aqueous sodium bicarbonate and extracted with ethyl acetate (×3). The combined organic layers were dried over sodium sulfate, filtered, and concentrated to afford crude (R*)-(2-allyl-2-azabicyclo[2.2.2]octan-1-yl)(phenyl)methanamine (0.122 g, 85 %) of an estimated 91% purity as a very light yellow clear oil that solidified to an oily semi-crystalline solid on standing. 1H NMR (300 MHz, chloroform-d) δ ppm 1.10-1.48 (m, 3 H), 1.50-1.70 (m, 6 H), 1.71-1.84 (m, 1 H), 1.94-2.07 (m, 1 H), 2.59 (dt, J=11.2, 2.5 Hz, 1 H), 3.01 (dd, J=14.1, 7.2 Hz, 1 H), 3.11 (d, J=10.7 Hz, 1 H), 3.67-3.80 (m, 1 H), 4.16 (s, 1 H), 5.12 (dd, J=10.1, 0.8 Hz, 1 H), 5.29 (dd, J=17.2, 1.4 Hz, 1 H), 5.83-6.01 (m, 1 H), 7.18-7.32 (m, 3 H), 7.32-7.39 (m, 2 H). ESI⁺ LCMS (M+H)⁺ 257.3.

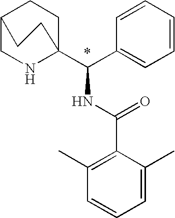

### Step D. Preparation of (R*)-N-(2-azabicyclo[2.2.2]octan-1-yl(phenyl)methyl)-2,6-dimethylbenzamide from (R*)-(2-allyl-2-azabicyclo[2.2.2]octan-1-yl)(phenyl)methanamine

Enantiopure (R*)-N-(2-azabicyclo[2.2.2]octan-1-yl(phenyl)methyl)-2,6-dimethylbenzamide was prepared from (R*)-(2-allyl-2-azabicyclo[2.2.2]octan-1-yl)(phenyl)methanamine using the procedure of Example 1, Step G, substituting (R*)-(2-allyl-2-azabicyclo[2.2.2]octan-1-yl)(phenyl)methanamine for (2-methyl-2-azabicyclo[2.2.2]octan-1-yl)(phenyl)methanamine and 2,6-dimethylbenzoic acid for 2,4-dichlorobenzoic acid. Additionally, purification was achieved using flash column chromatography (SiO₂, 0-60% ethyl acetate in hexanes). Amide coupling was then followed by the procedure of Example 6, Step B, using a 10 minute reaction time, and the reaction was worked-up as follows: the reaction mixture was poured into 1N aqueous hydrogen chloride and washed with ether. The ether layer was discarded, and the aqueous layer was basified with 50% aqueous sodium hydroxide. After extraction with ethyl acetate (×3), the combined organic layers were dried over sodium sulfate, filtered, and concentrated. The resulting residue was purified by flash column chromatography (SiO₂, 5% 2M ammonia in methanol in dichloromethane), and impure product fractions were repurified via preparative HPLC (C18, acetonitrile in water containing ammonium carbonate, pH 10). Pure product fractions from both purifications were then concentrated, and the resulting residues were combined to afford the desired product as a viscous oil. 1H NMR (500 MHz, chloroform-d) δ ppm 1.28-1.36 (m, 1 H), 1.46-1.54 (m, 1 H), 1.54-1.69 (m, 5 H), 1.71-1.78 (m, 1 H), 2.05-2.12 (m, 1 H), 2.24 (s, 6 H), 2.61 (s, 1 H), 2.86 (s, 2 H), 4.85 (d, J=8.2 Hz, 1 H), 7.00 (d, J=7.6 Hz, 2 H), 7.07-7.12 (m, 1 H), 7.14 (t, J=7.6 Hz, 1 H), 7.26-7.30 (m, 3 H), 7.31-7.36 (m, 2 H). ESI⁺ LCMS (M+H)⁺ 349.26.

Exemplary compounds of formula la, that can be made in accordance with the processes set forth herein include:

| | | | | | Mass |
|---|---|---|---|---|---|
| | | | | | spectroscopy |
| | | | | | mass ion(s), |
| | | IC₅₀ | Synthesis | | (HPLC retention |
| structure | Isomer | (uM) | Method | Name | time, method) |
| 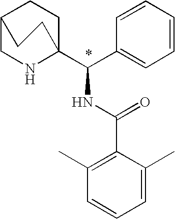 | R* | 0.00218 | 6, 7 | N-(2-azabicyclo[2.2.2]octan-1-yl(phenyl)methyl)-2,6-dimethylbenzamide | 349.26(0.88 min; MS2) |
| 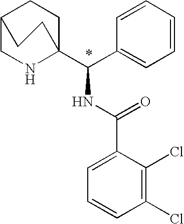 | R* | 0.0997 | 6 | N-(2-azabicyclo[2.2.2]octan-1-yl(phenyl)methyl)-2,3-dichlorobenzamide | 389.15(0.94 min; MS2) |
| 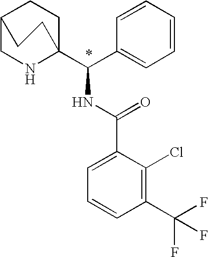 | R* | 0.455 | 6 | N-(2-azabicyclo[2.2.2]octan-1-yl(phenyl)methyl)-2-chloro-3-(trifluoromethyl)benzamide | 423.14(1.01 min; MS2) |
| 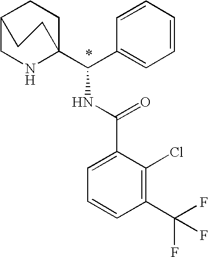 | S* | 0.198 | 6 | N-(2-azabicyclo[2.2.2]octan-1-yl(phenyl)methyl)-2-chloro-3-(trifluoromethyl)benzamide | 423.2(0.59 min; MS1) |
| 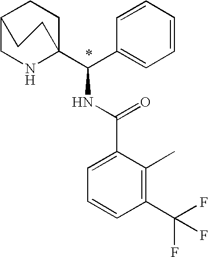 | R* | 0.0676 | 6 | N-(2-azabicyclo[2.2.2]octan-1-yl(phenyl)methyl)-2-methyl-3-(trifluoromethyl)benzamide | 403.19(1.02 min; MS2) |
| 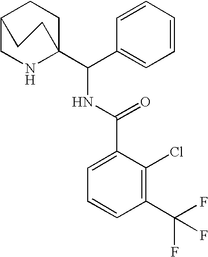 | RS | 0.258 | 5 | N-(2-azabicyclo[2.2.2]octan-1-yl(phenyl)methyl)-2-chloro-3-(trifluoromethyl)benzamide | 423.14, 425.15(9.08 min; MS4) |
| 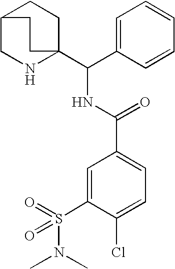 | RS | | | N-(2-azabicyclo[2.2.2]octan-1-yl(phenyl)methyl)-4-chloro-3-(N,N-dimethylsulfamoyl)benzamide | |
| 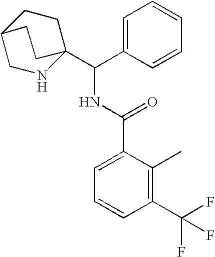 | RS | | | N-(2-azabicyclo[2.2.2]octan-1-yl(phenyl)methyl)-2-methyl-3-(trifluoromethyl)benzamide | |
| 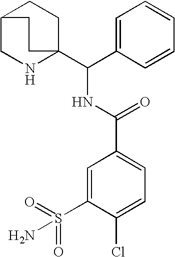 | RS | | | N-(2-azabicyclo[2.2.2]octan-1-yl(phenyl)methyl)-4-chloro-3-sulfamoylbenzamide | |
| 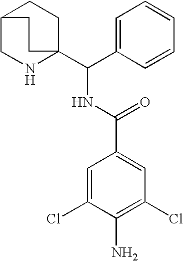 | RS | | | N-(2-azabicyclo[2.2.2]octan-1-yl(phenyl)methyl)-4-amino-3,5-dichlorobenzamide | |
| 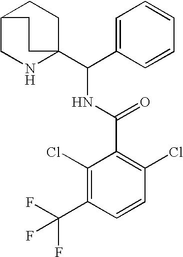 | RS | | | N-(2-azabicyclo[2.2.2]octan-1-yl(phenyl)methyl)-2,6-dichloro-3-(trifluoromethyl)benzamide | |
| 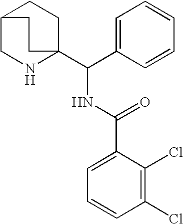 | RS | | | N-(2-azabicyclo[2.2.2]octan-1-yl(phenyl)methyl)-2,3-dichlorobenzamide | |
| 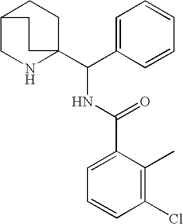 | RS | | | N-(2-azabicyclo[2.2.2]octan-1-yl(phenyl)methyl)-3-chloro-2-methylbenzamide | |
| 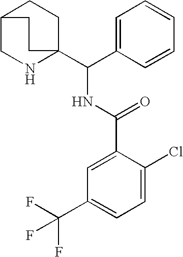 | RS | | | N-(2-azabicyclo[2.2.2]octan-1-yl(phenyl)methyl)-2-chloro-5-(trifluoromethyl)benzamide | |
| 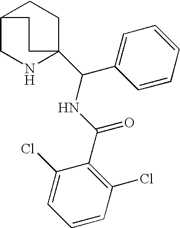 | RS | | | N-(2-azabicyclo[2.2.2]octan-1-yl(phenyl)methyl)-2,6-dichlorobenzamide | |
| 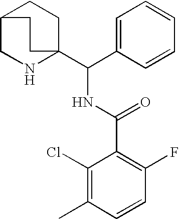 | RS | | | N-(2-azabicyclo[2.2.2]octan-1-yl(phenyl)methyl)-2-chloro-6-fluoro-3-methylbenzamide | |
| 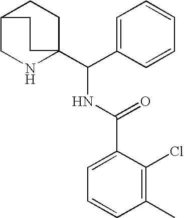 | RS | | | N-(2-azabicyclo[2.2.2]octan-1-yl(phenyl)methyl)-2-chloro-3-methylbenzamide | |
| 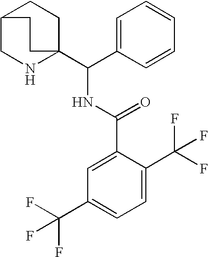 | RS | | | N-(2-azabicyclo[2.2.2]octan-1-yl(phenyl)methyl)-2,5-bis(trifluoromethyl)benzamide | |
| 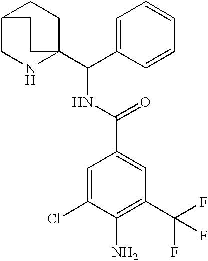 | RS | | | N-(2-azabicyclo[2.2.2]octan-1-yl(phenyl)methyl)-4-amino-3-chloro-5-(trifluoromethyl)benzamide | |
| 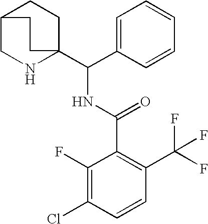 | RS | | | N-(2-azabicyclo[2.2.2]octan-1-yl(phenyl)methyl)-3-chloro-2-fluoro-6-(trifluoromethyl)benzamide | |
| 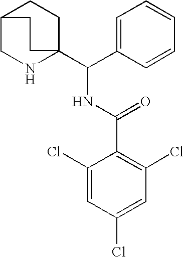 | RS | | | N-(2-azabicyclo[2.2.2]octan-1-yl(phenyl)methyl)-2,4,6-trichlorobenzamide | |
| 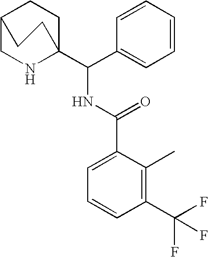 | RS | | | N-[(2-Aza-bicyclo[2.2.2]oct-1-yl)-phenyl-methyl]-2-methyl-3-trifluoromethyl-benzamide | |

What is claimed is:

1. A compound of Formula I: 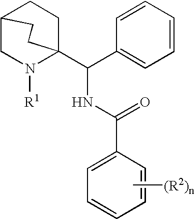
wherein:
R¹ is selected from H and C₁-C₆ alkyl;
Each R² is independently selected from halogen, —CN, C₂-C₆ alkenyl, C₂-C₆ alkynyl, C₃-C₆ cycloalkyl, —SO₂NR³R⁴, —NH₂, —S—C₁-C₆ alkyl, C₁-C₆ alkoxy, and C₁-C₆ alkyl, said C₁-C₆ alkyl and C₁-C₆ alkoxy being optionally substituted with one or more halogens;
R³ and R⁴ are each independently H or C₁-C₆ alkyl; and
n is 1, 2, or 3;
or a pharmaceutically acceptable salt thereof.

2. The compound of claim 1, wherein wherein R² is selected from —SO₂NR³R⁴, —NH₂, and C₁-C₆ alkyl optionally substituted with one or more halogens.

3. The compound of claim 1 which is substantially pure in the R configuration.

4. The compound of claim 1, wherein at least one R² is halogen.

5. The compound of claim 1, wherein at least one R² is C₁-C₆ alkyl, optionally substituted with one or more halogen.

6. The compound of claim 5,wherein at least one R² is methyl.

7. The compound of claim 5, wherein at least one R² is CF₃.

8. The compound of claim 1, wherein at least one R₂ is —S—C₁-C₆ alkyl.

9. The compound of claim 8, wherein at least one R² is —C₁-C₆-alkoxy optionally substituted with one or more halogens;

8. The compound of claim 1, wherein at least one R² is —SO₂NR³R⁴.

9. The compound of claim 1, wherein each of R³ and R⁴ are hydrogen.

10. The compound of claim 1, wherein each of R³ and R⁴ are C₁-C₆ alkyl.

11. The compound of claim 1, wherein n is 2.

12. The compound of claim 11, wherein one of R² is halogen and the other R² is selected from selected from halogen, —NH₂, —SO₂NR³R⁴, and C₁-C₆ alkyl optionally substituted with one or more halogens.

13. The compound of claim 12, wherein said other R² is C₁-C₆ alkyl optionally substituted with one or more halogens.

14. The compound of claim 12, wherein said one of R² is Cl, and the other R² is C₁-C₆ alkyl optionally substituted with one or more halogens.

15. The compound of claim 14, wherein said other R² is methyl.

16. The compound of claim 15, wherein said compound is selected from 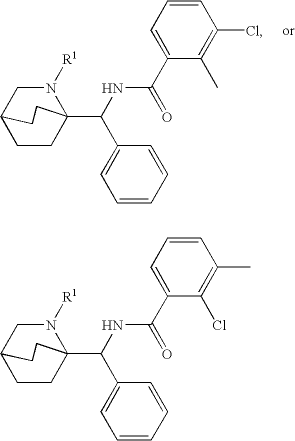
and pharmaceutically acceptable salts thereof.

17. The compound of claim 14, wherein said other R² is CF₃.

18. The compound of claim 17, wherein said compound is selected from 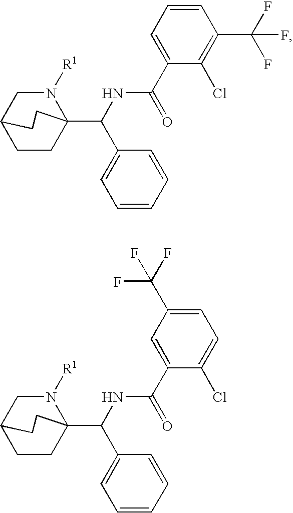
or a pharmaceutically acceptable salt thereof.

19. The compound of claim 12, wherein said other R² is —SO2NR³R⁴.

20. The compound of claim 19, wherein one of R² is Cl and the other R² is —SO₂NR³R⁴ where R³ and R⁴ are H.

21. The compound of claim 19, wherein one of R² is Cl and R³ and R⁴ are both C₁-C₆ alkyl optionally substituted with one or more halogens.

22. The compound of claim 21, wherein R³ and R⁴ are both methyl.

23. The compound of claim 19, wherein said compound is selected from 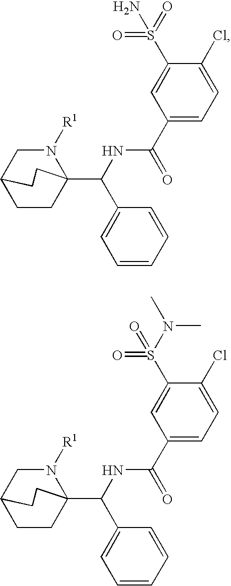
or a pharmaceutically acceptable salt thereof.

24. The compound of claim 12, wherein each R² is independently selected from the halogens.

25. The compound of claim 24, wherein at least one of said R² is Cl.

26. The compound of claim 25, where each R² is Cl.

27. The compound of claim 24, wherein said compound is selected from 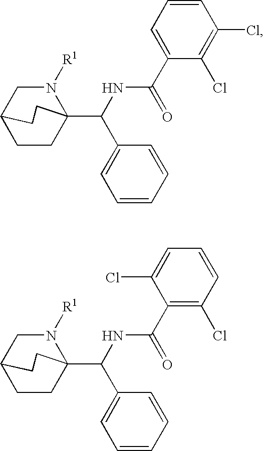
or a pharmaceutically acceptable salt thereof.

28. The compound of claim 11, wherein each R² is selected from —SO2NR³R⁴, —NH2, and C₁-C₆ alkyl optionally substituted with one or more halogens.

29. The compound of claim 28, wherein one of said R² is C₁-C₆ alkyl optionally substituted with one or more halogens.

30. The compound of claim 28, wherein each of said R² is C₁-C₆ alkyl optionally substituted with one or more halogens.

31. The compound of claim 30, wherein one of said R² is methyl.

32. The compound of claim 30, wherein one of said R² is CF₃.

33. The compound of claim 30, wherein one of said R² is methyl and said other R² is CF₃.

34. The compound of claim 30, wherein each of said R² is CF₃.

35. The compound of claim 28, wherein said compound is selected from 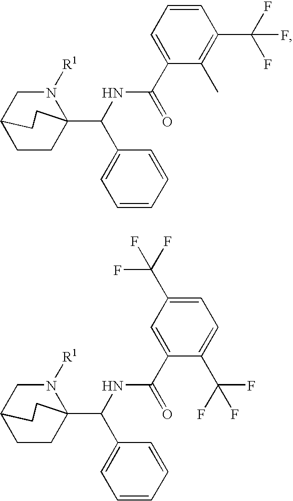
or a pharmaceutically acceptable salt thereof.

36. The compound of claim 1, wherein n is 3.

37. The compound of claim 36, wherein at least one of R² is halogen.

38. The compound of claim 36, wherein one of R² is halogen and each remaining R² is independently selected from NH₂ and CF₃.

39. The compound of claim 38, wherein said compound is example

40. The compound of claims 36, wherein at least two of R² are independently selected from the halogens.

41. The compound of claim 40, wherein at least two of R² are Cl.

42. The compound of claim 40, wherein the remaining R² is —NH₂ or —CF₃.

43. The compound of claim 40, wherein one of R² is Cl, one R² is F, and the remaining R² is C₁-C₆ alkyl optionally substituted with one or more halogen.

44. The compound of claim 43, where said remaining R² is methyl.

45. The compound of claim 40, wherein one of R² is Cl and one of R² is F.

46. The compound of claim 45, wherein the remaining R² is CF₃.

47. The compound of claim 36, wherein each R² is halogen.

48. The compound of claim 47 where each R² is Cl.

49. The compound of claim 36 selected from 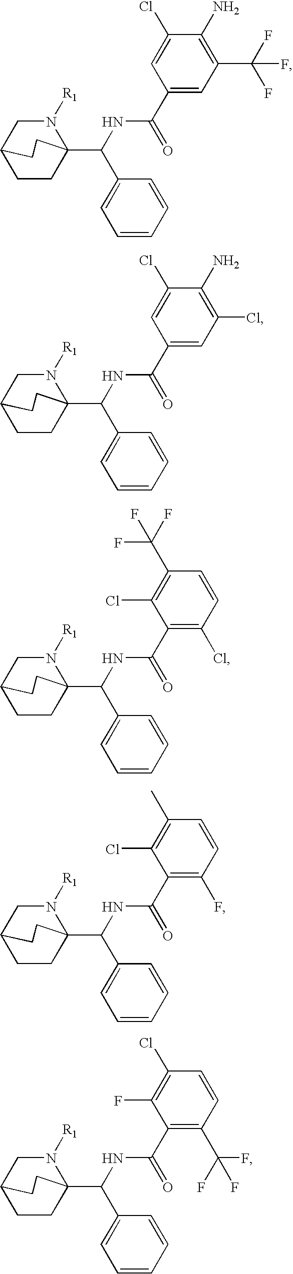 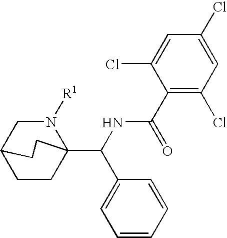
or a pharmaceutically acceptable salt thereof.

50. A compound of Formula Ia: 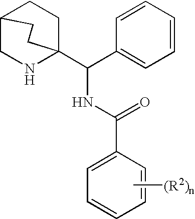
wherein
Each R² is independently selected from halogen, —CN, C₂-C₆ alkenyl, C₂-C₆ alkynyl, C₃-C₆ cycloalkyl, —SO₂NR³R⁴, —NH₂, —S—C₁-C₆ alkyl, C₁-C₆ alkoxy, and C₁-C₆ alkyl, said C₁-C₆ alkyl C₁-C₆ alkoxy being optionally substituted with one or more halogens;
R³ and R⁴ are each independently H or C₁-C₆ alkyl; and
n is 1, 2, or 3;
or a pharmaceutically acceptable salt thereof.

51. The compound of claim 50 selected from
N-(2-azabicyclo[2.2.2]octan-1-yl(phenyl)methyl)-2-chloro-3-(trifluoromethyl)benzamide;
N-(2-azabicyclo[2.2.2]octan-1-yl (phenyl)methyl)-4-chloro-3-(N,N-dimethylsulfamoyl)benzamide;
N-(2-azabicyclo[2.2.2]octan-1-yl (phenyl)methyl)-2-methyl-3-(trifluoromethyl)benzamide;
N-(2-azabicyclo[2.2.2]octan-1-yl(phenyl)methyl)-4-chloro-3-sulfamoylbenzamide;
N-(2-azabicyclo[2.2.2]octan-1-yl(phenyl)methyl)-4-amino-3,5-dichlorobenzamide;
N-(2-azabicyclo[2.2.2]octan-1-yl(phenyl)methyl)-2,6-dichloro-3-(trifluoromethyl)benzamide;
N-(2-azabicyclo[2.2.2]octan-1-yl(phenyl)methyl)-2,3-dichlorobenzamide;
N-(2-azabicyclo[2.2.2]octan-1-yl(phenyl)methyl)-3-chloro-2-methylbenzamide;
N-(2-azabicyclo[2.2.2]octan-1-yl(phenyl)methyl)-2-chloro-5-(trifluoromethyl)benzamide;
N-(2-azabicyclo[2.2.2]octan-1-yl(phenyl)methyl)-2,6-dichlorobenzamide;
N-(2-azabicyclo[2.2.2]octan-1-yl(phenyl)methyl)-2-chloro-6-fluoro-3-methylbenzamide;
N-(2-azabicyclo[2.2.2]octan-1-yl(phenyl)methyl)-2-chloro-3-methylbenzamide;
N-(2-azabicyclo[2.2.2]octan-1-yl(phenyl)methyl)-2,5-bis(trifluoromethyl)benzamide;
N-(2-azabicyclo[2.2.2]octan-1-yl(phenyl)methyl)-4-amino-3-chloro-5-(trifluoromethyl)benzamide;
N-(2-azabicyclo[2.2.2]octan-1-yl (phenyl)methyl)-3-chloro-2-fluoro-6-(trifluoromethyl)benzamide;
N-(2-azabicyclo[2.2.2]octan-1-yl(phenyl)methyl)-2,4,6-trichlorobenzamide;
or a pharmaceutically acceptable salt thereof.

52. The compound of claim 50 selected from
N-(2-azabicyclo[2.2.2]octan-1-yl(phenyl)methyl)-2-chloro-3-(trifluoromethyl)benzamide
N-(2-azabicyclo[2.2.2]octan-1-yl(phenyl)methyl)-2,3-dichlorobenzamide
N-(2-azabicyclo[2.2.2]octan-1-yl(phenyl)methyl)-2,3-dichlorobenzamide
N-(2-azabicyclo[2.2.2]octan-1-yl(phenyl)methyl)-2,6-dimethylbenzamide
N-(2-azabicyclo[2.2.2]octan-1-yl (phenyl)methyl)-2-methyl-3-(trifluoromethyl)benzamide
N-(2-azabicyclo[2.2.2]octan-1-yl (phenyl)methyl)-2-methyl-3-(trifluoromethyl)benzamide
N-(2-azabicyclo[2.2.2]octan-1-yl(phenyl)methyl)-4-chloro-3-(N, N-dimethylsulfamoyl)benzamide
N-(2-azabicyclo[2.2.2]octan-1-yl(phenyl)methyl)-4-chloro-3-sulfamoylbenzamide
N-(2-azabicyclo[2.2.2]octan-1-yl(phenyl)methyl)-4-amino-3,5-dichlorobenzamide
N-(2-azabicyclo[2.2.2]octan-1-yl(phenyl)methyl)-2,6-dichloro-3-(trifluoromethyl)benzamide
N-(2-azabicyclo[2.2.2]octan-1-yl(phenyl)methyl)-3-chloro-2-methylbenzamide
N-(2-azabicyclo[2.2.2]octan-1-yl(phenyl)methyl)-2-chloro-5-(trifluoromethyl)benzamide
N-(2-azabicyclo[2.2.2]octan-1-yl(phenyl)methyl)-2,6-dichlorobenzamide
N-(2-azabicyclo[2.2.2]octan-1-yl (phenyl)methyl)-2-chloro-6-fluoro-3-methylbenzamide
N-(2-azabicyclo[2.2.2]octan-1-yl(phenyl)methyl)-2-chloro-3-methylbenzamide
N-(2-azabicyclo[2.2.2]octan-1-yl(phenyl)methyl)-2,5-bis(trifluoromethyl)benzamide
N-(2-azabicyclo[2.2.2]octan-1-yl(phenyl)methyl)-4-amino-3-chloro-5-(trifluoromethyl)benzamide
N-(2-azabicyclo[2.2.2]octan-1-yl (phenyl)methyl)-3-chloro-2-fluoro-6-(trifluoromethyl)benzamide
N-(2-azabicyclo[2.2.2]octan-1-yl(phenyl)methyl)-2,4,6-trichlorobenzamide
or a pharmaceutically acceptable salt thereof.

53. A compound of Formula Ib: 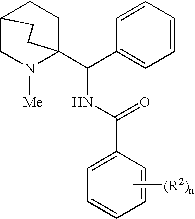
wherein:
Each R² is independently selected from halogen, —CN, C₂-C₆ alkenyl, C₂-C₆ alkynyl, C₃-C₆ cycloalkyl, —SO₂NR³R⁴, —NH₂, —S—C₁-C₆ alkyl, C₁-C₆ alkoxy, and C₁-C₆ alkyl, said C₁-C₆ alkyl and C₁-C₆ alkoxy being optionally substituted with one or more halogens;
R³ and R⁴ are each independently H or C₁-C₆ alkyl; and
n is 1, 2, or 3;
or a pharmaceutically acceptable salt thereof.

54. The compound of claim 53 selected from
2-chloro-N-((2-methyl-2-azabicyclo[2.2.2]octan-1-yl)(phenyl)methyl)-3-(trifluoromethyl)benzamide;
4-chloro-3-(N,N-dimethylsulfamoyl)-N-((2-methyl-2-azabicyclo[2.2.2]octan-1-yl)(phenyl)methyl)benzamide;
2-methyl-N-((2-methyl-2-azabicyclo[2.2.2]octan-1-yl)(phenyl)methyl)-3-(trifluoromethyl)benzamide;
4-chloro-N-((2-methyl-2-azabicyclo[2.2.2]octan-1-yl)(phenyl)methyl)-3-sulfamoylbenzamide;
4-amino-3,5-dichloro-N-((2-methyl-2-azabicyclo[2.2.2]octan-1-yl)(phenyl)methyl)benzamide;
2,6-dichloro-N-((2-methyl-2-azabicyclo[2.2.2]octan-1-yl)(phenyl)methyl)-3-(trifluoromethyl)benzamide;
2,3-dichloro-N-((2-methyl-2-azabicyclo[2.2.2]octan-1-yl)(phenyl)methyl)benzamide;
3-chloro-2-methyl-N-((2-methyl-2-azabicyclo[2.2.2]octan-1-yl)(phenyl)methyl)benzamide;
2-chloro-N-((2-methyl-2-azabicyclo[2.2.2]octan-1-yl)(phenyl)methyl)-5-(trifluoromethyl)benzamide;
2,6-dichloro-N-((2-methyl-2-azabicyclo[2.2.2]octan-1-yl)(phenyl)methyl)benzamide;
2-chloro-6-fluoro-3-methyl-N-((2-methyl-2-azabicyclo[2.2.2]octan-1-yl)(phenyl)methyl)benzamide;
2-chloro-3-methyl-N-((2-methyl-2-azabicyclo[2.2.2]octan-1-yl)(phenyl)methyl)benzamide;
N-((2-methyl-2-azabicyclo[2.2.2]octan-1-yl)(phenyl)methyl)-2,5-bis(trifluoromethyl)benzamide;
4-amino-3-chloro-N-((2-methyl-2-azabicyclo[2.2.2]octan-1-yl)(phenyl)methyl)-5-(trifluoromethyl)benzamide;
3-chloro-2-fluoro-N-((2-methyl-2-azabicyclo[2.2.2]octan-1-yl)(phenyl)methyl)-6-(trifluoromethyl)benzamide;
2,4,6-trichloro-N-((2-methyl-2-azabicyclo[2.2.2]octan-1-yl)(phenyl)methyl)benzamide;
or a pharmaceutically acceptable salt thereof.

55. The compound of claim 53 selected from
4-Fluoro-2-methoxy-N-[(2-methyl-2-aza-bicyclo[2.2.2]oct-1-yl)-phenyl-methyl]-benzamide;
N-[(2-Methyl-2-aza-bicyclo[2.2.2]oct-1-yl)-phenyl-methyl]-4-trifluoromethyl-benzamide;
2-Amino-6-chloro-N-[(S)-(2-methyl-2-aza-bicyclo[2.2.2]oct-1-yl)-phenyl-methyl]-benzamide;
2-Chloro-6-methyl-N-[(2-methyl-2-aza-bicyclo[2.2.2]oct-1-yl)-phenyl-methyl]-benzamide;
2,3-Dichloro-N-[(2-methyl-2-aza-bicyclo[2.2.2]oct-1-yl)-phenyl-methyl]-benzamide;
3-Fluoro-2-methyl-N-[(2-methyl-2-aza-bicyclo[2.2.2]oct-1-yl)-phenyl-methyl]-benzamide;
N-[(2-Methyl-2-aza-bicyclo[2.2.2]oct-1-yl)-phenyl-methyl]-2-methylsulfanyl-benzamide;
2-Amino-6-chloro-N-[(2-methyl-2-aza-bicyclo[2.2.2]oct-1-yl)-phenyl-methyl]-benzamide;
2,6-Dimethoxy-N-[(2-methyl-2-aza-bicyclo[2.2.2]oct-1-yl)-phenyl-methyl]-benzamide;
2-Chloro-3-fluoro-N-[(R)-(2-methyl-2-aza-bicyclo[2.2.2]oct-1-yl)-phenyl-methyl]-benzamide;
2,4-Dichloro-N-[(2-methyl-2-aza-bicyclo[2.2.2]oct-1-yl)-phenyl-methyl]-benzamide;
2-Methyl-N-[(2-methyl-2-aza-bicyclo[2.2.2]oct-1-yl)-phenyl-methyl]-benzamide;
2-Chloro-4-fluoro-N-[(R)-(2-methyl-2-aza-bicyclo[2.2.2]oct-1-yl)-phenyl-methyl]-benzamide;
2-Chloro-N-[(R)-(2-methyl-2-aza-bicyclo[2.2.2]oct-1-yl)-phenyl-methyl]-3-trifluoromethylbenzamide;
2-Amino-6-chloro-N-[(R)-(2-methyl-2-aza-bicyclo[2.2.2]oct-1-yl)-phenyl-methyl]-benzamide;
2,4-Dichloro-N-[(R)-(2-methyl-2-aza-bicyclo[2.2.2]oct-1-yl)-phenyl-methyl]-benzamide;
2-Chloro-4-fluoro-N-[(2-methyl-2-aza-bicyclo[2.2.2]oct-1-yl)-phenyl-methyl]-benzamide;
2-Bromo-N-[(2-methyl-2-aza-bicyclo[2.2.2]oct-1-yl)-phenyl-methyl]-benzamide;
2-Chloro-3-fluoro-N-[(2-methyl-2-aza-bicyclo[2.2.2]oct-1-yl)-phenyl-methyl]-benzamide;
2-Bromo-6-chloro-N-[(2-methyl-2-aza-bicyclo[2.2.2]oct-1-yl)-phenyl-methyl]-benzamide;
N-[(2-Methyl-2-aza-bicyclo[2.2.2]oct-1-yl)-phenyl-methyl]-2-trifluoromethoxy-benzamide;
2-Bromo-3-fluoro-N-[(2-methyl-2-aza-bicyclo[2.2.2]oct-1-yl)-phenyl-methyl]-benzamide;
2-Chloro-N-[(S)-(2-methyl-2-aza-bicyclo[2.2.2]oct-1-yl)-phenyl-methyl]-3-trifluoromethylbenzamide;
2-Chloro-5-methoxy-N-[(2-methyl-2-aza-bicyclo[2.2.2]oct-1-yl)-phenyl-methyl]-benzamide;
2-Chloro-N-[(2-methyl-2-aza-bicyclo[2.2.2]oct-1-yl)-phenyl-methyl]-3-trifluoromethyl-benzamide;
2,6-Dichloro-N-[(2-methyl-2-aza-bicyclo[2.2.2]oct-1-yl)-phenyl-methyl]-benzamide;
2-Fluoro-N-[(2-methyl-2-aza-bicyclo[2.2.2]oct-1-yl)-phenyl-methyl]-6-trifluoromethyl-benzamide;
2-Chloro-N-[(2-methyl-2-aza-bicyclo[2.2.2]oct-1-yl)-phenyl-methyl]-benzamide;
N-[(2-Methyl-2-aza-bicyclo[2.2.2]oct-1-yl)-phenyl-methyl]-2,5-bis-trifluoromethyl-benzamide;
2,3-Dichloro-N-[(S)-(2-methyl-2-aza-bicyclo[2.2.2]oct-1-yl)-phenyl-methyl]-benzamide;
N-[(2-Aza-bicyclo[2.2.2]oct-1-yl)-phenyl-methyl]-2-chloro-3-trifluoromethyl-benzamide;
2-Methoxy-N-[(2-methyl-2-aza-bicyclo[2.2.2]oct-1-yl)-phenyl-methyl]-benzamide;
4-Fluoro-2-methoxy-N-[(2-methyl-2-aza-bicyclo[2.2.2]oct-1-yl)-phenyl-methyl]-benzamide;
N-[(2-Methyl-2-aza-bicyclo[2.2.2]oct-1-yl)-phenyl-methyl]-4-trifluoromethyl-benzamide;
2-Amino-6-chloro-N-[(S)-(2-methyl-2-aza-bicyclo[2.2.2]oct-1-yl)-phenyl-methyl]-benzamide;
or a pharmaceutically acceptable salt thereof.

56. The compound of claim 53 selected from:
2,3-dichloro-N-((2-methyl-2-azabicyclo[2.2.2]octan-1-yl)(phenyl)methyl)benzamide;
(R)-2,3-dichloro-N-((2-methyl-2-azabicyclo[2.2.2]octan-1-yl)(phenyl)methyl)benzamide;
(S)-2,3-dichloro-N-((2-methyl-2-azabicyclo[2.2.2]octan-1-yl)(phenyl)methyl)benzamide;
2,4-dichloro-N-((2-methyl-2-azabicyclo[2.2.2]octan-1-yl)(phenyl)methyl)benzamide;
(R)-2,4-dichloro-N-((2-methyl-2-azabicyclo[2.2.2]octan-1-yl)(phenyl)methyl)benzamide;
2,6-dichloro-N-((2-methyl-2-azabicyclo[2.2.2]octan-1-yl)(phenyl)methyl)benzamide;
2,6-dichloro-N-((2-methyl-2-azabicyclo[2.2.2]octan-1-yl)(phenyl)methyl)benzamide;
(R)-2,6-dichloro-N-((2-methyl-2-azabicyclo[2.2.2]octan-1-yl)(phenyl)methyl)benzamide;
2,6-dimethoxy-N-((2-methyl-2-azabicyclo[2.2.2]octan-1-yl)(phenyl)methyl)benzamide;
2,6-dimethyl-N-((2-methyl-2-azabicyclo[2.2.2]octan-1-yl)(phenyl)methyl)benzamide;
(R)-2,6-dimethyl-N-((2-methyl-2-azabicyclo[2.2.2]octan-1-yl)(phenyl)methyl)benzamide;
2-amino-6-chloro-N-((2-methyl-2-azabicyclo[2.2.2]octan-1-yl)(phenyl)methyl)benzamide;
(R)-2-amino-6-chloro-N-((2-methyl-2-azabicyclo[2.2.2]octan-1-yl)(phenyl)methyl)benzamide;
2-bromo-3-fluoro-N-((2-methyl-2-azabicyclo[2.2.2]octan-1-yl)(phenyl)methyl)benzamide;
2-bromo-6-chloro-N-((2-methyl-2-azabicyclo[2.2.2]octan-1-yl)(phenyl)methyl)benzamide;
2-bromo-6-methyl-N-((2-methyl-2-azabicyclo[2.2.2]octan-1-yl)(phenyl)methyl)benzamide;
2-bromo-N-((2-methyl-2-azabicyclo[2.2.2]octan-1-yl)(phenyl)methyl)benzamide;
2-chloro-3-fluoro-N-((2-methyl-2-azabicyclo[2.2.2]octan-1-yl)(phenyl)methyl)benzamide;
(R)-2-chloro-3-fluoro-N-((2-methyl-2-azabicyclo[2.2.2]octan-1-yl)(phenyl)methyl)benzamide;
2-chloro-4-fluoro-N-((2-methyl-2-azabicyclo[2.2.2]octan-1-yl)(phenyl)methyl)benzamide;
(R)-2-chloro-4-fluoro-N-((2-methyl-2-azabicyclo[2.2.2]octan-1-yl)(phenyl)methyl)benzamide;
2-chloro-5-methoxy-N-((2-methyl-2-azabicyclo[2.2.2]octan-1-yl)(phenyl)methyl)benzamide;
2-chloro-6-methoxy-N-((2-methyl-2-azabicyclo[2.2.2]octan-1-yl)(phenyl)methyl)benzamide;
(R)-2-chloro-6-methoxy-N-((2-methyl-2-azabicyclo[2.2.2]octan-1-yl)(phenyl)methyl)benzamide;
2-chloro-6-methyl-N-((2-methyl-2-azabicyclo[2.2.2]octan-1-yl)(phenyl)methyl)benzamide;
2-chloro-N-((2-methyl-2-azabicyclo[2.2.2]octan-1-yl)(phenyl)methyl)benzamide;
2-chloro-N-((2-methyl-2-azabicyclo[2.2.2]octan-1-yl)(phenyl)methyl)-3-(trifluoromethyl)benzamide;
(R)-2-chloro-N-((2-methyl-2-azabicyclo[2.2.2]octan-1-yl)(phenyl)methyl)-3-(trifluoromethyl)benzamide;
2-fluoro-N-((2-methyl-2-azabicyclo[2.2.2]octan-1-yl)(phenyl)methyl)-3-(trifluoromethyl)benzamide;
2-fluoro-N-((2-methyl-2-azabicyclo[2.2.2]octan-1-yl)(phenyl)methyl)-6-(trifluoromethyl)benzamide;
2-methyl-N-((2-methyl-2-azabicyclo[2.2.2]octan-1-yl)(phenyl)methyl)-3-(trifluoromethyl)benzamide;
2-methyl-N-((2-methyl-2-azabicyclo[2.2.2]octan-1-yl)(phenyl)methyl)benzamide;
3-fluoro-2-methyl-N-((2-methyl-2-azabicyclo[2.2.2]octan-1-yl)(phenyl)methyl)benzamide;
N-((2-methyl-2-azabicyclo[2.2.2]octan-1-yl)(phenyl)methyl)-2-(methylthio)benzamide;
N-((2-methyl-2-azabicyclo[2.2.2]octan-1-yl)(phenyl)methyl)-2-(trifluoromethoxy)benzamide;
N-((2-methyl-2-azabicyclo[2.2.2]octan-1-yl)(phenyl)methyl)-2,5-bis(trifluoromethyl)benzamide;
N-((2-methyl-2-azabicyclo[2.2.2]octan-1-yl)(phenyl)methyl)-2,5-bis(trifluoromethyl)benzamide;
(R)-2-ethoxy-N-((2-methyl-2-azabicyclo[2.2.2]octan-1-yl)(phenyl)methyl)benzamide;
(R)-2-ethyl-N-((2-methyl-2-azabicyclo[2.2.2]octan-1-yl)(phenyl)methyl)benzamide;
(R)-N-((2-methyl-2-azabicyclo[2.2.2]octan-1-yl)(phenyl)methyl)-2-(trifluoromethyl)benzamide;
(S)-2-chloro-N-((2-methyl-2-azabicyclo[2.2.2]octan-1-yl)(phenyl)methyl)-3-(trifluoromethyl)benzamide;
4-chloro-3-(N,N-dimethylsulfamoyl)-N-((2-methyl-2-azabicyclo[2.2.2]octan-1-yl)(phenyl)methyl)benzamide;
4-chloro-N-((2-methyl-2-azabicyclo[2.2.2]octan-1-yl)(phenyl)methyl)-3-sulfamoylbenzamide;
4-amino-3,5-dichloro-N-((2-methyl-2-azabicyclo[2.2.2]octan-1-yl)(phenyl)methyl)benzamide;
2,6-dichloro-N-((2-methyl-2-azabicyclo[2.2.2]octan-1-yl)(phenyl)methyl)-3-(trifluoromethyl)benzamide;
3-chloro-2-methyl-N-((2-methyl-2-azabicyclo[2.2.2]octan-1-yl)(phenyl)methyl)benzamide;
2-chloro-N-((2-methyl-2-azabicyclo[2.2.2]octan-1-yl)(phenyl)methyl)-5-(trifluoromethyl)benzamide;
2-chloro-6-fluoro-3-methyl-N-((2-methyl-2-azabicyclo[2.2.2]octan-1-yl)(phenyl)methyl)benzamide;
2-chloro-3-methyl-N-((2-methyl-2-azabicyclo[2.2.2]octan-1-yl)(phenyl)methyl)benzamide;
4-amino-3-chloro-N-((2-methyl-2-azabicyclo[2.2.2]octan-1-yl)(phenyl)methyl)-5-(trifluoromethyl)benzamide;
3-chloro-2-fluoro-N-((2-methyl-2-azabicyclo[2.2.2]octan-1-yl)(phenyl)methyl)-6-(trifluoromethyl)benzamide;
2,4,6-trichloro-N-((2-methyl-2-azabicyclo[2.2.2]octan-1-yl)(phenyl)methyl)benzamide;
or a pharmaceutically acceptable salt thereof.

57. A pharmaceutical composition comprising a therapeutically effective amount of a compound according to claim 1 and a pharmaceutically acceptable carrier or diluent.

58. A pharmaceutical composition comprising a therapeutically effective amount of a compound according to claim 50 and a pharmaceutically acceptable carrier or diluent.

59. A pharmaceutical composition comprising a therapeutically effective amount of a compound according to claim 53 and a pharmaceutically acceptable carrier or diluent.

60. A method of using a compound of claim 1 for the treatment of psychoses.

61. A method of claim 60, wherein said psychoses is selected from schizophrenia, bi-polar disorder, mania and manic depression, and anxiety disorders.

62. A method for treating psychoses comprising administering a therapeutically effective amount of a compound according to claim 1 to a patient in need thereof.

63. A method of claim 62, wherein said psychoses is schizophrenia.

64. The use of a compound according to claim 1 in the manufacture of a medicament for the treatment or prophylaxis of psychoses or other cognitive disorder.
